(19) United States
(12) Patent Application Publication
(10) Pub. No.: US 2025/0113995 A1
(43) Pub. Date: Apr. 10, 2025

(54) INFORMATION PROCESSING APPARATUS, METHOD OF CONTROLLING INFORMATION PROCESSING APPARATUS, AND STORAGE MEDIUM

(71) Applicant: CANON KABUSHIKI KAISHA, Tokyo (JP)

(72) Inventors: YOSHIHIKO IWASE, Kanagawa (JP); ETSURO WADA, Kanagawa (JP); HIROKI UCHIDA, Tokyo (JP); RIUMA TAKAHASHI, Tokyo (JP)

(21) Appl. No.: 18/918,502

(22) Filed: Oct. 17, 2024

Related U.S. Application Data
(63) Continuation of application No. PCT/JP2023/013457, filed on Mar. 31, 2023.

(30) Foreign Application Priority Data
Apr. 19, 2022 (JP) 2022-068833
Apr. 19, 2022 (JP) 2022-068834

(51) Int. Cl. A61B 3/10; A61B 3/00; A61B 3/14

(52) U.S. Cl. CPC A61B 3/102; A61B 3/0041; A61B 3/14

(57) ABSTRACT

One of information processing apparatuses according to the technique disclosed herein includes an examination unit, a storage unit, and a control unit. The examination unit is configured to examine an eye of an examinee. The storage unit is configured to store a plurality of examination protocols, each of the plurality of examination protocols including a plurality of examinations. The control unit is configured to control a display unit such that information that represents a plurality of examinations included in each of at least two examination protocols among the plurality of examination protocols stored is displayed on a protocol-by-protocol basis for the at least two examination protocols, and is configured to control the examination unit in accordance with one examination protocol selected according to an instruction given from an examiner from among the at least two examination protocols.

### CROSS-REFERENCE TO RELATED APPLICATIONS

This application is a Continuation of International Patent Application No. PCT/JP2023/013457, filed on Mar. 31, 2023, which claims the benefit of Japanese Patent Application No. 2022-068833, filed on Apr. 19, 2022, and Japanese Patent Application No. 2022-068834, filed on Apr. 19, 2022, both of which are hereby incorporated by reference herein in their entirety.

### TECHNICAL FIELD

The technique disclosed herein relates to an information processing apparatus, a method of controlling an information processing apparatus, and a non-transitory computer-readable storage medium.

### BACKGROUND ART

An apparatus for acquiring a two-dimensional fundus image of the subject's eye (hereinafter will be referred to as “fundus camera apparatus”) and an apparatus for acquiring a tomographic image of the subject's eye by using an optical coherence tomography (OCT) method based on low coherence light (hereinafter will be referred to as “OCT apparatus”) are practically used as examples of an ophthalmic apparatus.

These apparatuses perform image capturing after making alignment adjustments and other necessary adjustments of the apparatus and the subject's eye. An ophthalmic apparatus that has an AUTO function of making these adjustments automatically have recently been developed. By using the AUTO function, the user is able to capture an image of the subject's eye easily with the ophthalmic apparatus, without any need for performing complex adjusting operations.

Japanese Patent Laid-Open No. 2018-139716 discloses a technique that makes it possible to, in an ophthalmic apparatus storing an examination protocol including a plurality of imaging conditions, generate an examination protocol easily by providing a user interface so as to assist the user who is unskilled in imaging or in maneuvering the apparatus.

Japanese Patent Laid-Open No. 2014-39870 discloses an optical image measurement apparatus that has a function of acquiring a tomographic image of a fundus and acquiring a fundus image. The optical image measurement apparatus disclosed in Japanese Patent Laid-Open No. 2014-39870 further has a function of automating tasks needed at the time of imaging such as automatic imaging, automatic focusing, and the like. In the optical image measurement apparatus disclosed in Japanese Patent Laid-Open No. 2014-39870, any manner of scanning is selected in advance from among a plurality of manners of scanning of signal light for fundus scanning. After that, if the function of automating tasks needed at the time of imaging is ON, the acquisition of a tomographic image of a fundus and the acquisition of a fundus image are performed automatically on the basis of the selected manner of scanning.

### CITATION LIST

### Patent Literature

PTL 1 Japanese Patent Laid-Open No. 2018-139716
PTL 2 Japanese Patent Laid-Open No. 2014-39870

In ophthalmic apparatuses of related art, a list of examination protocols is displayed at the time of imaging. When this list is displayed, in the related art, for example, at the time of selecting an examination protocol, it is sometimes difficult to know a combination of imaging operations included in the examination protocol and the sequential order thereof, and imaging conditions such as a condition as to whether automatic imaging is performed or manual imaging is performed. Moreover, for example, since the related art does not provide display that shows the progress status of the examination protocol while imaging is being performed, it is sometimes difficult to know the progress of the examination protocol. Furthermore, a task of image-captured examination confirmation could be burdensome to the operator because it is not easy to confirm all of image-captured examinations at one time.

### SUMMARY OF INVENTION

One of the techniques disclosed herein aims to enhance the usability of an ophthalmic apparatus.

Note that the aim described above does not imply any limitation. Producing working effects derived by each configuration to be described in Description of Embodiments below and unable to be obtained from the related art can be positioned as another non-limiting aim of the present disclosure.

One of information processing apparatuses according to the technique disclosed herein includes an examination unit, a storage unit, and a control unit. The examination unit is configured to examine an eye of an examinee. The storage unit is configured to store a plurality of examination protocols, each of the plurality of examination protocols including a plurality of examinations. The control unit is configured to control a display unit such that information that represents a plurality of examinations included in each of at least two examination protocols among the plurality of examination protocols stored is displayed on a protocol-by-protocol basis for the at least two examination protocols, and is configured to control the examination unit in accordance with one examination protocol selected according to an instruction given from an examiner from among the at least two examination protocols.

Further features of the present invention will become apparent from the following description of exemplary embodiments with reference to the attached drawings.

### BRIEF DESCRIPTION OF DRAWINGS

FIG. 1 is a diagram that illustrates a schematic configuration example of an ophthalmic apparatus according to a first embodiment.

FIG. 2 is a diagram that illustrates a schematic configuration example of a control unit according to the first embodiment.

FIG. 3 is a diagram that illustrates an example of screen transitions according to the first embodiment.

FIG. 4 is a diagram that illustrates an example of a flowchart of an examination protocol according to the first embodiment.

FIG. 5 is a diagram that illustrates an example of patient screen display according to the first embodiment.

FIG. 6 is a diagram that illustrates an example of imaging screen display according to the first embodiment.

FIG. 7 is a diagram that illustrates an example of examination protocol selection screen display according to the first embodiment.

FIG. 8 is a diagram that illustrates an example of examination protocol selection screen display according to the first embodiment.

FIG. 9A is a diagram that illustrates an example of progress dialogue screen display according to the first embodiment.

FIG. 9B is a diagram that illustrates an example of progress dialogue screen display according to the first embodiment.

FIG. 10 is a diagram that illustrates an example of progress dialogue screen display according to the first embodiment.

FIG. 11 is a diagram that illustrates an example of a flowchart of an examination protocol according to a second embodiment.

FIG. 12 is a diagram that illustrates an example of imaging screen display according to the second embodiment.

FIG. 13 is a diagram that illustrates an example of imaging screen display according to the second embodiment.

FIG. 14 is a diagram that illustrates an example of a flowchart of an examination protocol according to a third embodiment.

FIG. 15 is a diagram that illustrates an example of imaging screen display according to the third embodiment.

FIG. 16 is a diagram that illustrates an example of imaging screen display according to the third embodiment.

FIG. 17 is a diagram that illustrates an example of imaging screen display according to the third embodiment.

FIG. 18 is a diagram that illustrates an example of imaging screen display according to the third embodiment.

FIG. 19 is a diagram that illustrates an example of confirmation screen display according to a fourth embodiment.

FIG. 20 is a diagram that illustrates an example of confirmation screen display according to the fourth embodiment.

FIG. 21A is a diagram that illustrates an example of confirmation screen display according to the fourth embodiment.

FIG. 21B is a diagram that illustrates an example of confirmation screen display according to the fourth embodiment.

FIG. 22 is a diagram that illustrates an example of confirmation screen display according to the fourth embodiment.

FIG. 23 is a diagram that illustrates an example of confirmation screen display (enlarged display) according to the fourth embodiment.

FIG. 24 is a diagram that illustrates an example of confirmation screen display according to the fourth embodiment.

FIG. 25 is a diagram that illustrates an example of confirmation screen display according to the fourth embodiment.

FIG. 26 is a diagram that illustrates an example of confirmation screen display according to a fifth embodiment.

FIG. 27 is a diagram that illustrates an example of confirmation screen display according to the fifth embodiment.

FIG. 28 is a diagram that illustrates an example of confirmation screen display according to the fifth embodiment.

FIG. 29 is a diagram that illustrates an example of confirmation screen display according to the fourth embodiment.

FIG. 30 is a diagram that illustrates an example of confirmation screen display according to the fourth embodiment.

FIG. 31 is a diagram that illustrates an example of confirmation screen display according to the fourth embodiment.

### DESCRIPTION OF EMBODIMENTS

With reference to the drawings, exemplary embodiments for implementing the technique disclosed herein will now be described in detail. However, dimensions, materials, shapes, relative positions of components, and the like to be described in the embodiments below are not restrictive, and can be changed depending on the configuration of an apparatus to which the technique disclosed herein is applied or depending on various conditions. In the drawings, the same reference signs are used for components that are identical to, or functionally similar to, one another.

### First Embodiment

In the present embodiment, an ophthalmic apparatus that performs both OCT imaging and fundus imaging using visible light will be described as an example of an ophthalmic apparatus according to the technique disclosed herein. Protocol names only are displayed by an optical image measurement apparatus according to related art at the time of examination protocol selection, and there is thus no way to know a combination of examinations included in a certain examination protocol until this examination protocol is selected. For this reason, in related art, usability is low for an operator who is unskilled in imaging or in maneuvering the apparatus.

In view of this, the present embodiment aims to enhance the usability of an ophthalmic apparatus. In an ophthalmic apparatus according to the present embodiment, an examination protocol(s) defining a series of control procedures including both OCT imaging and fundus imaging using visible light is stored, and, when an operator makes a choice from among a plurality of examination protocols, examination conditions included in the examination protocols are presented so as to enable the operator to easily select one of the examination protocols to be executed.

Furthermore, an ophthalmic apparatus according to the present embodiment enables the operator to know the progress status of the examinations by furnishing the operator with information about the progress status of the examination protocol. Therefore, the operator can operate a plurality of apparatuses simultaneously, and, even if the operator leaves the apparatus unattended temporarily, the operator can know the progress of the examinations upon coming back.

An ophthalmic system according to the present embodiment may be configured as follows. An ophthalmic apparatus (complex examination unit) according to the technique disclosed herein is connected to a network, receives an order (written examination instructions) from a personal computer of a doctor, executes an examination of the subject's eye(s) in accordance with the order, and then transmits the results of the examination to the personal computer of the doctor.

### Configuration

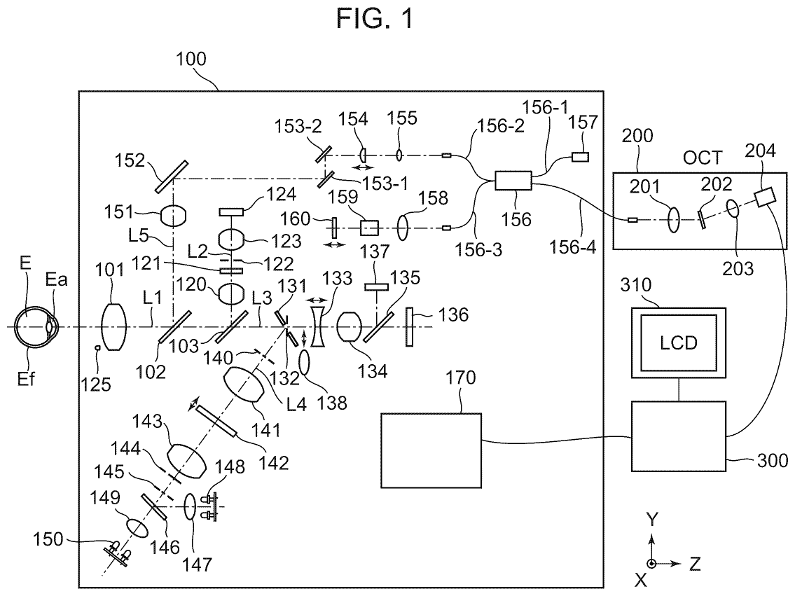

FIG. 1 is a diagram that illustrates a schematic configuration example of an ophthalmic apparatus according to the present embodiment and various optical systems included in the ophthalmic apparatus. An ophthalmic apparatus according to the present embodiment includes an optical head unit 100, a spectrometer 200, a control unit 300, a display unit 310, an input unit 340, and a voice output unit 350. The configuration of the optical head unit 100, the spectrometer 200, and the control unit 300 will now be described in order.

### Configuration of Optical Head Unit 100 and Spectrometer 200

The optical head unit 100 is configured as a measuring optical system for capturing an image of an anterior eye segment Ea of a subject's eye E, and a two-dimensional frontal image of a fundus Ef of the subject's eye E and a tomographic image thereof. Various optical systems arranged inside the optical head unit 100 are described below.

In the optical head unit 100, an objective lens 101 facing the subject's eye E is provided. A first dichroic mirror 102 and a second dichroic mirror 103 each of which functions as an example of an optical path splitter are disposed on an optical axis L1 of the objective lens 101. Due to the presence of these dichroic mirrors, an optical path leading from the objective lens 101 branches into an optical path of an anterior eye segment observation system (optical axis L2), an optical path of a fundus imaging system (optical axis L3), and an optical path of an OCT optical system (optical axis L5), based on each wavelength band.

A lens 120, a prism 121, an iris 122, a lens 123, and an image sensor 124 are disposed on the optical axis L2 lying in the reflection direction of the second dichroic mirror 103. The image sensor 124 is a monochrome sensor that has sensitivity in an infrared region. These optical members, etc. disposed on the optical axis L2 constitute the anterior eye segment observation system for observing the anterior eye segment Ea. An anterior eye segment observation light source 125 is disposed near the objective lens 101. The anterior eye segment observation light source 125 illuminates the anterior eye segment of the subject's eye E by using infrared light.

The image sensor 124 is connected to the control unit 300. Based on a signal outputted from the image sensor 124, the control unit 300 is capable of generating an anterior eye segment observation image and outputting the anterior eye segment observation image to the display unit 310 or causing a storage unit 302 to store the anterior eye segment observation image.

A pierced mirror 131, an imaging iris 132, a focus lens 133, an image forming lens 134, a third dichroic mirror 135, and an image sensor 136 are disposed on the optical axis L3 lying in the transmission direction of the second dichroic mirror 103. The pierced mirror 131 has an opening at its center. The focus lens 133 is capable of moving on the optical axis L3 by being driven by a non-illustrated driver such as a motor controlled by the control unit 300. The control unit 300 is capable of adjusting the focus of light passing along the optical path of the fundus imaging system by causing the focus lens 133 to move in the optical axis direction by controlling the driver.

Due to the presence of the third dichroic mirror 135, the optical path on the optical axis L3 branches into an optical path leading to the image sensor 136 and an optical path leading to a fixation lamp 137, based on each wavelength band. The image sensor 136 is a frontal fundus image sensor that is disposed in the transmission direction of the third dichroic mirror 135, has visible light sensitivity and infrared light sensitivity, and serves for dual purposes of moving-picture imaging and still-picture imaging for observation. The fixation lamp 137 is disposed in the reflection direction of the third dichroic mirror 135, and generates visible light for visual fixation of the subject. Other optical members such as a non-illustrated iris for cutting off light that is not needed for fundus imaging may be provided on the optical path of the fundus imaging system.

A cornea baffle 140, a relay lens 141, a focus index unit 142, a lens 143, and a ring slit 144 are disposed in this order on an optical axis L4 lying in the reflection direction of the pierced mirror 131. The cornea baffle 140 has a light-shielding point at its center. The ring slit 144 has a ring-shaped slit opening.

The focus index unit 142 is an optical member that provides an index for focus adjustment using the focus lens 133, and, in the present embodiment, emits split emission lines as an example of the index. The focus index unit 142 according to the present embodiment includes a split index member that is movable along the optical axis L4 in linkage with the focus lens 133. The split index member is configured to be able to be inserted into, and removed from, the optical path of the optical axis L4 by being driven by a non-illustrated driver such as a motor controlled by the control unit 300.

The split emission lines emitted by the focus index unit 142 pass through the relay lens 141 and are reflected toward the second dichroic mirror 103 by the pierced mirror 131. The split emission lines having been reflected by the pierced mirror 131 pass through the second dichroic mirror 103, the first dichroic mirror 102, and the objective lens 101 and are projected onto the fundus Ef of the subject's eye E. The control unit 300 is capable of calculating an amount of focus deviation by detecting the positions of the split emission lines from a fundus observation image.

A crystalline lens baffle 145 that serves as a light-shielding member having a light-shielding point and a dichroic mirror 146 that has characteristics of allowing infrared light to pass and reflecting visible light are further disposed on the optical axis L4. A condenser lens 147 and a white LED light source 148 are disposed in the reflection direction of the dichroic mirror 146. The white LED light source 148 is an imaging light source in which a plurality of white LEDs configured to emit visible light is disposed. A condenser lens 149 and an infrared LED light source 150 are disposed in the transmission direction of the dichroic mirror 146. The infrared LED light source 150 is an observation light source in which a plurality of infrared LEDs configured to emit infrared stationary light is disposed. The drive operation of the white LED light source 148 and the infrared LED light source 150 is controlled by the control unit 300.

The objective lens 101, the dichroic mirror 146, optical members disposed therebetween, and the condenser lenses 147 and 149 constitute an illuminating optical system for illuminating the fundus Ef. It is possible to illuminate the fundus Ef of the subject's eye E by using light coming via the illuminating optical system from the white LED light source 148 or the infrared LED light source 150.

A lens 151, a mirror 152, an OCTX scanner 153-1, an OCTY scanner 153-2, a focus lens 154, and a lens 155 are disposed on the optical axis L5 lying in the reflection direction of the first dichroic mirror 102. The OCTX scanner 153-1 and the OCTY scanner 153-2 are deflectors such as, for example, galvanometer mirrors, and function as a scanning unit configured to scan measurement light on the fundus Ef of the subject's eye E. The position near the center of the OCTX scanner 153-1 and the OCTY scanner 153-2 is optically conjugate to the position of the pupil of the subject's eye E. Though the optical path between the OCTX scanner 153-1 and the OCTY scanner 153-2 lies on the sheet face of FIG. 1, actually, the direction of this optical path is perpendicular to the sheet face. An MEMS mirror or the like that is capable of deflecting light in a two-dimensional direction with a single mirror configuration may be used as the scanning unit configured to scan measurement light.

In the present embodiment, the OCTX scanner 153-1 is capable of scanning measurement light in the X direction, and the OCTY scanner 153-2 is capable of scanning measurement light in the Y direction, which is orthogonal to the X direction. In the present embodiment, the X direction is defined as a main scanning direction, the Y direction is defined as a sub-scanning direction, and an example of performing 3D scan will be described; however, the scanning direction is not limited thereto. The main scanning direction and the sub-scanning direction in 3D scan or raster scan may be any directions as long as they intersect with each other. For example, the Y direction may be the main scanning direction, and the X direction may be the sub-scanning direction. Oblique directions that intersect with each other and include X-directional and Y-directional components may be the main scanning direction and the sub-scanning direction. The pattern of scanning is not limited to 3D scan. For example, radial scan, cross scan, circle scan, raster scan, or the like may be adopted.

The focus lens 154 is capable of moving on the optical axis L5 by being driven by a non-illustrated driver such as a motor controlled by the control unit 300. The control unit 300 is capable of adjusting the focus of measurement light passing along the optical path of the OCT coherency system by causing the focus lens 154 to move in the optical axis direction by controlling the driver.

The focus adjustment of measurement light is performed such that measurement light emitted from a fiber end of an optical fiber 156-2 that behaves as a light source forms an image on the fundus Ef. The fiber end of the optical fiber 156-2 is optically conjugate to the fundus Ef of the subject's eye E. The focus lens 154, which functions as a focus adjuster, is disposed between the fiber end serving as the light source of the measurement light and the OCTX scanner 153-1 and the OCTY scanner 153-2 functioning as the scanning unit. Therefore, by performing a focus adjustment using the focus lens 154, it is possible to form an image of the measurement light emitted from the fiber end on the fundus Ef of the subject's eye E and to send return light coming back from the fundus Ef to the optical fiber 156-2 efficiently.

Next, an optical path leading from a measurement light source 157 and the configuration of a referential optical system and the spectrometer 200 will now be described. The measurement light source 157 is a light source that emits light for obtaining measurement light that enters a measuring optical path (the optical path of the OCT coherency system). In the present embodiment, an SLD (Super Luminescent Diode), which is a typical low-coherence light source, is used as the measurement light source 157. The center wavelength of light emitted from the measurement light source 157 is 880 nm, and the wavelength width thereof is approximately 60 nm. The wavelength width is an important parameter because it has an influence on the resolution in the optical axis direction of a tomographic image that is obtained. Though an SLD is selected in this example, the type of the light source may be any type as long as low-coherence light can be emitted, for example, ASE (Amplified Spontaneous Emission) or the like may be used. As for the center wavelength of the measurement light, for example, with eye measurement in mind, near infrared light may be used. It is necessary to allocate wavelength differences to some extent between the wavelength used for the optical path of the OCT coherency system (the optical axis L5), the wavelength used for the optical path of the anterior eye segment observation system (the optical axis L2), and the wavelength used for the optical path of the fundus imaging system (the optical axis L3). In the present embodiment, in view of this, the above wavelength is selected as the wavelength of the SLD.

The light emitted from the measurement light source 157 is guided to an optical coupler 156 through an optical fiber 156-1. The light having been guided to the optical coupler 156 is split by the optical coupler 156 into measurement light that goes toward the optical fiber 156-2 and reference light that goes toward an optical fiber 156-3. The optical coupler 156 functions as an example of a splitter configured to split the light coming from the measurement light source 157 into the measurement light and the reference light. The optical fibers 156-1 to 4 are single-mode optical fibers connected integrally to the optical coupler 156.

In the present embodiment, the measurement light in the OCT optical system is emitted from the fiber end of the optical fiber 156-2 serving as a light source. The measurement light is applied to the fundus Ef of the subject's eye E, which is the target of imaging, via the optical path of the OCT optical system described above, and goes back along the same optical path due to retinal reflection and scattering to reach the optical coupler 156 again.

On the other hand, the reference light goes through the optical fiber 156-3, a lens 158, and a dispersion-compensating glass 159 inserted for matching dispersion of the measurement light and the reference light, reaches a reference mirror 160, and is reflected thereat. The reference light having been reflected by the reference mirror 160 returns along the same optical path to reach the optical coupler 156 again.

The reference light having reached the optical coupler 156 again is merged with the measurement light (return light) by the optical coupler 156. When the optical path length of the measurement light and the optical path length of the reference light become almost equal to each other, this merging causes interference of the light waves. The reference mirror 160 is held such that its position can be adjusted in the optical axis direction of the reference light by being driven by a non-illustrated driver such as a motor controlled by the control unit 300. Using such a driver makes it possible to adjust the optical path length of the reference light to the optical path length of the measurement light that varies depending on the subject's eye E. The interfering light having been obtained in this way is guided to the spectrometer 200 through the optical fiber 156-4.

A lens 201, a diffraction grating 202, a lens 203, and a line sensor 204 are provided in the spectrometer 200. The interfering light having gone out from the optical fiber 156-4 turns into substantially parallel light through the lens 201 and is thereafter optically separated by the diffraction grating 202; then, an image is formed on the line sensor 204 by the lens 203. Each element in the line sensor 204 outputs a signal according to received light (interfering signal) to the control unit 300.

The control unit 300 is capable of acquiring the signal outputted from the line sensor 204 via its image acquisition unit 304 to be described later, and performing sampling at predetermined timing and performing predetermined signal processing thereon by its processing unit 305, thereby generating a tomographic image.

The measurement light source 157, the optical coupler 156, the optical fibers 156-1 to 4, the lens 158, the dispersion-compensating glass 159, the reference mirror 160, and the spectrometer 200 constitute a Michelson interferometer. Though a Michelson interferometer is used as the interferometer in the present embodiment, a Mach-Zehnder interferometer may be used instead.

A head driving unit 170 is further provided in the optical head unit 100. The head driving unit 170 includes three non-illustrated motors controlled by the control unit 300. By controlling the driving of the head driving unit 170, the control unit 300 is capable of moving the optical head unit 100 in three-dimensional directions (X, Y, and Z). By this means, the control unit 300 can perform an alignment of the optical head unit 100 with the subject's eye E. Configuration of Control Unit 300

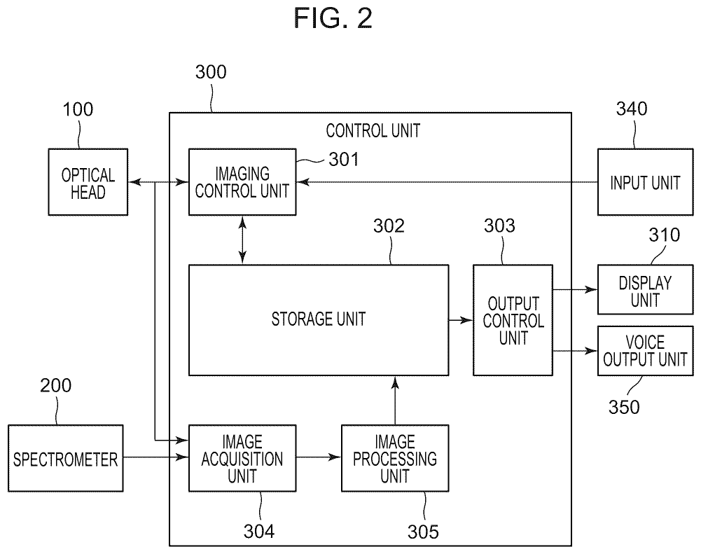

Next, with reference to FIG. 2, a schematic configuration of the control unit 300 will now be described. The control unit 300 is connected to the optical head unit 100, the spectrometer 200, and the display unit 310, and can control them. The control unit 300 is further connected to the input unit 340, and can receive an instruction from an operator in accordance with operation of the input unit 340 by the operator. An imaging control unit 301, the storage unit 302, the image acquisition unit 304, and the processing unit 305 are provided in the control unit 300.

The imaging control unit 301 can exchange information with the optical head unit 100, the storage unit 302, the processing unit 305, and the input unit 340. Based on input signals sent from the input unit 340, signals sent from the optical head unit 100 and the processing unit 305, information stored in the storage unit 302, and the like, the imaging control unit 301 can control various components of the optical head unit 100.

The imaging control unit 301 can calculate an amount of focus deviation on the basis of split emission lines shown in a fundus observation image by driving the focus index unit 142, and can obtain diopter scale information about the subject's eye E on the basis of the deviation amount. Any known method may be used for calculating the amount of focus deviation. Any known method may be used for acquiring the diopter scale information.

The image acquisition unit 304 can acquire signals outputted from the image sensor 124 and the image sensor 136 of the optical head unit 100. The image acquisition unit 304 can acquire an interfering signal outputted from the line sensor 204 of the spectrometer 200.

The processing unit 305 can generate an anterior eye segment observation image on the basis of a signal acquired from the image sensor 124. The processing unit 305 can generate a frontal fundus image on the basis of a signal acquired from the image sensor 136. Furthermore, the processing unit 305 can generate a tomographic image on the basis of an interfering signal acquired from the line sensor 204. Any known method may be used for generating various images by the processing unit 305. Specific control of the optical head unit 100 by the control unit 300 is described below.

In capturing an anterior eye segment observation image, the imaging control unit 301 causes the anterior eye segment observation light source 125 to emit light. The image sensor 124 receives light reflected from the anterior eye segment Ea of the subject's eye E, and outputs a signal that is based on the received light to the control unit 300. The image acquisition unit 304 acquires the signal outputted from the image sensor 124. The processing unit 305 generates an anterior eye segment observation image on the basis of the acquired signal. The generated anterior eye segment observation image can be stored into the storage unit 302. The generated anterior eye segment observation image can be displayed on the display unit 310 by an output control unit 303.

In capturing a fundus observation image using infrared light by means of a fundus camera, the imaging control unit 301 causes the infrared LED light source 150 to perform light emission. The image sensor 136 receives infrared light reflected from the fundus Ef of the subject's eye E, and outputs a signal that is based on the received infrared light to the control unit 300. The image acquisition unit 304 acquires the signal outputted from the image sensor 136. The processing unit 305 generates a fundus observation image on the basis of the acquired signal.

The imaging control unit 301 drives the focus index unit 142. Based on split emission lines shown in the fundus observation image, the processing unit 305 acquires diopter scale information about the subject's eye E. Based on the acquired diopter scale information, the imaging control unit 301 drives the focus lens 133. When adjusting a focus to a wider diopter range, the imaging control unit 301 can perform the focus adjustment by using a non-illustrated driver to insert a diopter compensation lens 138 onto, and remove it from, the optical axis L3, and by driving the focus lens 133 on the basis of the diopter scale information.

The generated fundus observation image can be used for fundus observation and for tracking the subject's eye E to be described later. The generated fundus observation image can be displayed on the display unit 310 by the output control unit 303. The generated fundus observation image can be stored into the storage unit 302.

In capturing the fundus image by means of the fundus camera, the imaging control unit 301 performs a focus adjustment by using the focus lens 133. Specifically, diopter scale information is calculated while applying aberration correction that is based on differences between light source wavelengths to the diopter scale information about the subject's eye E acquired using infrared light in the fundus observation image capturing. Based on the calculated diopter scale information, the imaging control unit 301 changes the position of the focus lens 133. After that, the imaging control unit 301 causes the white LED light source 148 to emit visible light. The image sensor 136 receives the visible light reflected from the fundus Ef of the subject's eye E, and outputs a signal that is based on the received light to the control unit 300. The image acquisition unit 304 acquires the signal outputted from the image sensor 136. The processing unit 305 generates a frontal fundus image on the basis of the signal acquired from the image sensor 136.

In capturing a tomographic image, based on diopter scale information with application of aberration correction that is based on differences between light source wavelengths to the diopter scale information acquired using the focus index unit 142, the imaging control unit 301 drives the focus lens 154. Based on an imaging range obtained using the fundus observation image in accordance with the operator's instruction and information about the position of the focus lens 133, the imaging control unit 301 calculates scanning information that includes the direction of deflection and the width of deflection by the OCTX scanner 153-1 and the OCTY scanner 153-2. The imaging control unit 301 sends the calculated scanning information to the OCTX scanner 153-1 and the OCTY scanner 153-2 for scanning of measurement light on the fundus Ef of the subject's eye E.

The line sensor 204 outputs, to the control unit 300, an interfering signal that is based on the return light of the received measurement light. The image acquisition unit 304 acquires the interfering signal outputted from the line sensor 204. The processing unit 305 performs Fourier transformation on the signal acquired from the line sensor 204, and transforms the Fourier-transformed data into luminance information or density information, thereby acquiring a tomographic image in the depth direction (Z direction) of the subject's eye E. Acquiring an image (information) in the depth direction at one point of the subject's eye E by using the scan scheme described above is referred to as “A scan”, and a tomographic image obtained by this scan is referred to as “A scan image”.

The time taken for image processing of a tomographic image by the processing unit 305 is 20 microseconds or less. In the present embodiment, the data of the line sensor 204 is transmitted at intervals of 20 microseconds; therefore, if the next data is acquired at the line sensor 204 while the processing unit 305 performs image processing, it is possible to perform A scan at intervals of 20 microseconds. The time pertaining to the processing described above is not limited to this example, and may be changed depending on a desired configuration.

The control unit 300 can acquire a plurality of A scan images by causing the scanning unit to scan measurement light for A scan in a predetermined traversing direction on the fundus Ef. The processing unit 305 can form a tomographic image along the predetermined traversing direction from the plurality of A scan images and the scanning information. For example, a tomographic image on an X-Z plane can be obtained by performing scanning in the X direction, and a tomographic image on a Y-Z plane can be obtained by performing scanning in the Y direction. The above-described scan scheme of scanning measurement light in a predetermined traversing direction (main scanning direction) on the subject's eye E is referred to as “B scan”, and a tomographic image obtained by this scan is referred to as “B scan image”.

Moreover, it is possible to acquire a plurality of B scan images by repeating scanning in the main scanning direction while moving the position of scanning by the scanning unit in a predetermined direction (sub-scanning direction) in an imaging range on the subject's eye E. For example, it is possible to obtain three-dimensional information in an X-Y-Z space by repeating B scan on an X-Z plane while moving the scanning position in the Y direction by the imaging control unit 301. The data configured using the acquired plurality of B scan images is referred to as three-dimensional data. The processing unit 305 can generate a three-dimensional tomographic image of the subject's eye E from the three-dimensional data. This scan scheme is referred to as “C scan”, and a three-dimensional tomographic image obtained by this scan is referred to as “C scan image”. The processing unit 305 can acquire an En-Face image (frontal image) of the fundus Ef, too; this can be done by performing projection or summation of information in at least a part of the depth range of the three-dimensional information.

The storage unit 302 pre-stores various kinds of information, etc. needed for processing at the control unit 300. In addition, various images generated by the processing unit 305 are stored in the storage unit 302, for example, an anterior eye segment observation image, an infrared fundus observation image, a fundus image, a B scan image that is a tomographic image, three-dimensional data, an OCT frontal fundus image, and the like of the subject's eye E. Moreover, examination sequences each defining a series of control procedures involving execution of examination more than once, image analysis results, imaging conditions at the time of image acquisition, patient information about the subject's eye E, and the like are stored in the storage unit 302. Furthermore, various programs for controlling the above-described anterior eye segment observation image capturing, the above-described infrared fundus observation image capturing and fundus image capturing using the fundus camera, and the above-described tomographic image capturing, and a program for controlling each component, may also be stored in the storage unit 302. The storage unit 302 may be any kind of storage medium such as, for example, an optical disc or a memory.

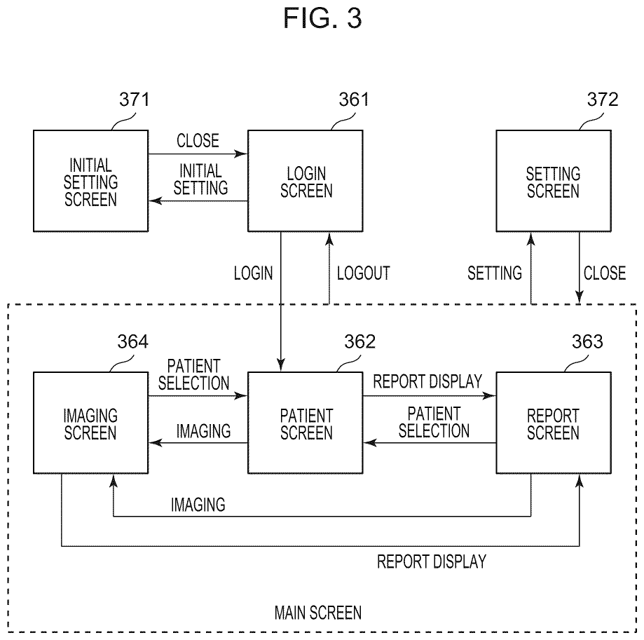

The output control unit 303 is connected to the display unit 310 such as a display device to display an anterior eye segment observation image, an infrared fundus observation image, a fundus image, a B scan image that is a tomographic image, three-dimensional data, and an En-Face image that are stored in the storage unit 302. An example of display by the display unit 310 is illustrated in FIG. 3.

The control unit 300 may be configured using, for example, a general-purpose computer. The control unit 300 may be configured using a dedicated ophthalmic-apparatus-only computer. The control unit 300 includes a non-illustrated processor and a non-illustrated storage medium including a memory such as an optical disc or a ROM (Read Only Memory). The processor may be a CPU (Central Processing Unit), an MPU (Micro Processing Unit), or the like. The processor is not limited to a CPU or an MPU. A GPU (Graphics Processing Unit), an FPGA (Field-Programmable Gate Array), or the like may be used instead. Each component of the control unit 300 other than the storage unit 302 may be configured as a software module that is run by the processor such as a CPU, an MPU, a GPU, or the like. Alternatively, each of these components may be configured as a circuit that fulfills a particular function of an ASIC or the like, an independent device, or the like. The processor or the circuit may include a digital signal processor (DSP), a data flow processor (DFP), or a neural processing unit (NPU).

The display unit 310 is configured using any kind of monitor, and displays various images, patient information, and the like under the control of the output control unit 303. The input unit 340 includes any kind of input device such as a mouse, a keyboard, etc. Though the control unit 300, the display unit 310, the input unit 340, and the like are provided separately in the present embodiment, a part or a whole of them may be configured integrally. For example, a touch panel may be used as the display unit 310 and the input unit 340. In the present embodiment, the display unit 310 and the input unit 340 are described as a touch panel.

### Examination Flow

Next, with reference to FIGS. 3 to 10, an examination flow according to the present embodiment will now be described. In the present embodiment, an examination flow of capturing images of both eyes through examinations included in an examination protocol successively, upon selection of the examination protocol, will be described.

FIG. 3 is an example of a screen transition chart according to the present embodiment. In FIG. 3, a login screen 361 is a screen that is displayed first on the display unit 310. An operator can perform various kinds of operation after performing a login via the login screen 361. An initial setting screen 371 is a screen on which initial settings for various kinds of operation can be performed after performing a login via the login screen, and on which settings different from those performed on a setting screen 372 are performed. Performing settings just once suffices on the initial setting screen 371. Items whose setting change is less frequent can be set on this screen. Examples of them include login user management, data storage destination management, license management, and the like. On the setting screen 372, items for which setting change is wanted depending on each individual operator and depending on the timing of imaging, etc., such as during-imaging settings and display settings, can be set. Examples of them include internal fixation lamp display settings, shortcut key settings, voice output settings, and the like. A patient screen 362 is a screen for displaying a list of pieces of patient data. A report screen 363 is a screen for displaying examination (captured) images of each individual patient (subject) and displaying the results of analyzing various images. An imaging screen 364 is a screen for performing imaging by operating the ophthalmic apparatus and displaying examination (captured) images.

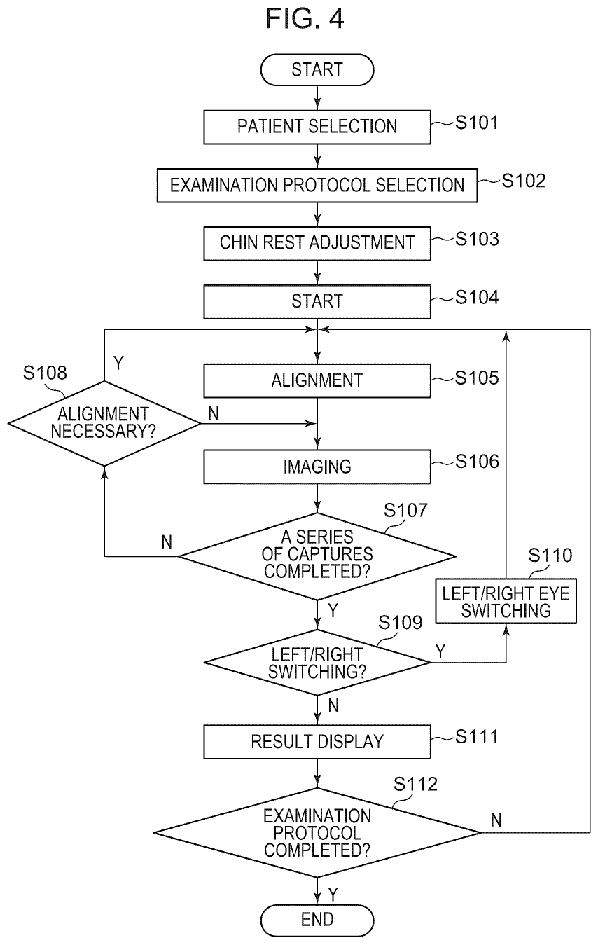

Next, with reference to an examination flowchart of FIG. 4, an examination example according to the present embodiment will now be described.

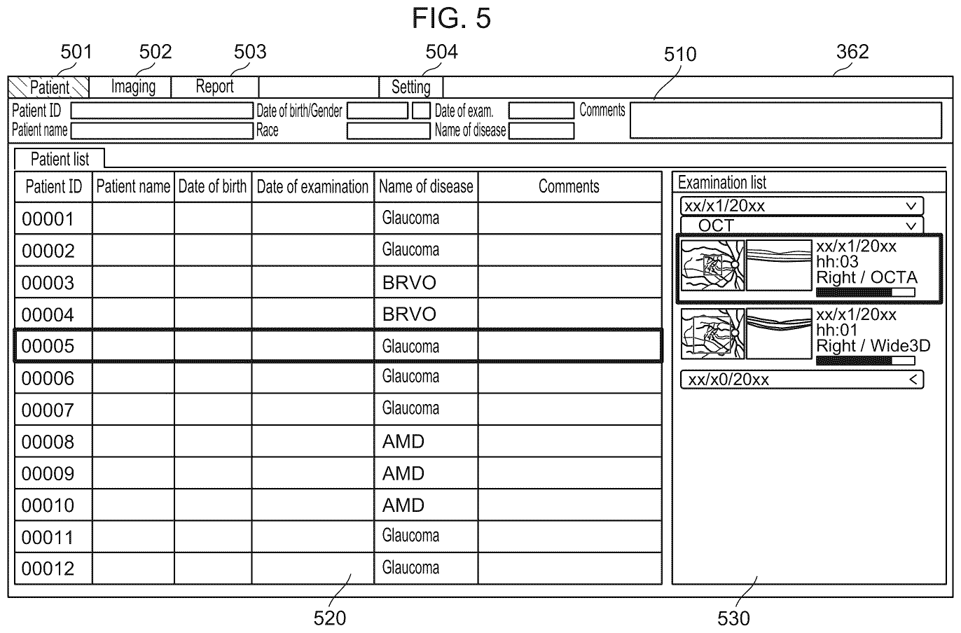

After performing a login via a non-illustrated login screen, an operator performs new registration or selection of a patient on the patient screen 362 (step S101). This will now be described with reference to FIG. 5. FIG. 5 illustrates an example of the patient screen 362 according to the present embodiment. In the patient screen 362, numeral 501 denotes a patient tab, numeral 502 denotes an imaging tab, numeral 503 denotes a report tab, numeral 504 denotes a setting tab, and oblique lines in the patient tab 501 represent that the patient screen 362 is in an active state. The patient screen 362 includes a patient information field 510, a patient list 520, and an examination list 530. On the patient screen 362, the operator enters new patient information in the patient information field 510 to make a transition to the imaging screen 364 or selects patient data from the patient list 520 to make a transition to the imaging screen 364. In entering new patient information, a barcode reader may be used for registering the information. If the display unit 310 is a touch panel, a keyboard may be displayed on the display unit 310, and the operator may touch the keyboard to enter the patient information. In the present embodiment, an existing patient ID 00005 is selected to make a transition to the imaging screen 364.

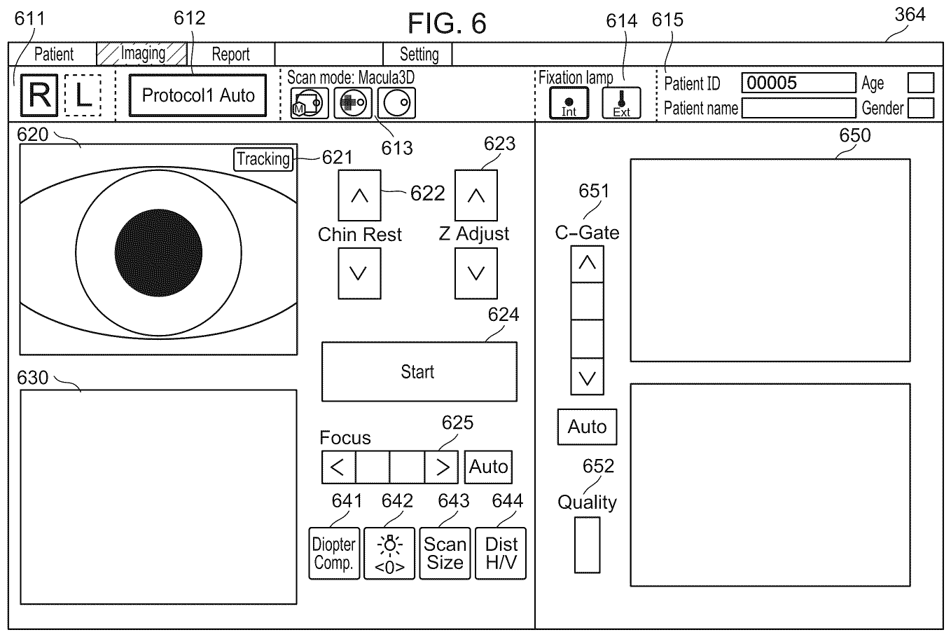

Next, with reference to FIG. 6, the imaging screen 364 will now be described. FIG. 6 illustrates a screen example of a state before a start of OCT imaging with the chin of the subject on a chin rest. Numeral 611 denotes a left/right eye button that indicates which one of the left eye and the right eye is to be image captured. A state of image capturing the right eye is illustrated in FIG. 6. With this button, it is possible to give an instruction to the apparatus for switching the target of imaging between the left eye and the right eye. Numeral 612 denotes an examination protocol selection button. It is possible to, via this button, display and choose an examination protocol according to which the imaging is to be performed. A detailed explanation will be given later in step S102. Numeral 613 denotes icons of examinations included in the examination protocol. Numeral 614 denotes fixation lamp buttons for an internal fixation lamp and an external fixation lamp. Though a single kind of button is shown for each of the internal fixation lamp and the external fixation lamp in 614, another button for changing the size of the internal fixation lamp may be provided. Buttons for switching between blinking and lighting of the fixation lamp may be provided. Switching between blinking and lighting may be performed by touching a button more than once. Numeral 615 denotes a patient information display section. Information related to a patient such as a patient ID, a patient name, age, gender, etc. is displayed in this section.

Numeral 620 denotes an anterior eye segment observation image display section. Numeral 621 denotes a tracking state of the anterior eye segment. The operator can control the X-Y position of the optical head unit 100 by touching the image in the anterior eye segment observation image display section 620. Instead of controlling the X-Y position by touching the anterior eye segment image, a non-illustrated adjuster (for example, a button) may be displayed, and the X-Y position may be controlled by operating the adjuster. Numeral 622 denotes a chin rest adjuster. Numeral 623 denotes a Z adjuster for controlling the Z position of the optical head unit 100. Numeral 625 denotes a focus adjuster. Both a manual focus adjustment and an automatic focus adjustment are available. Numeral 624 denotes a start button. The operator can give an instruction for starting imaging by operating this button.

Numeral 630 denotes a fundus observation image display section. Upon the start, this section displays a fundus observation image, and, in a case where OCT imaging is performed, superimposes an imaging range and a scanning pattern on the fundus observation image. Numeral 641 denotes a diopter compensation adjustment button. Numeral 642 denotes an observation light amount adjustment button. Numeral 643 denotes an OCT scan size adjustment button. Numeral 644 denotes an OCT scan interval adjustment button.

Numeral 650 denotes an OCT tomographic image display section. Upon the start, this section displays one tomographic image of horizontal scan and one tomographic image of vertical scan. Numeral 651 denotes a coherence gate adjuster. Both a manual coherence gate adjustment and an automatic coherence gate adjustment are available. Numeral 652 denotes a quality evaluation indicator for an OCT tomographic image.

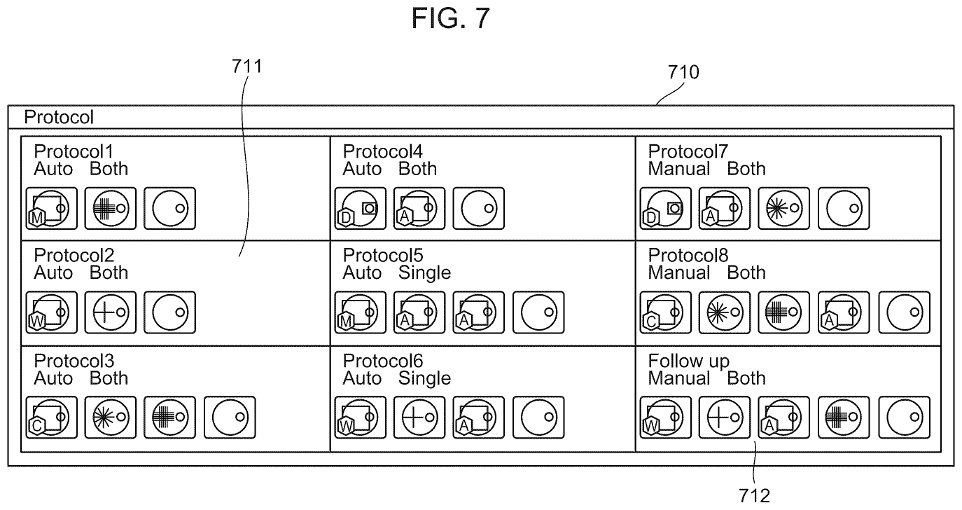

Next, examination protocol selection on the imaging screen 364 in step S102 will now be described. At the time of a transition to the imaging screen 364, display appears in a state in which a defined examination protocol (Protocol 1) is selected. When a follow-up observation is performed, the same examination protocol as the previous one may be set. In the present embodiment, with reference to FIG. 7, an example of selecting Protocol 2 as an examination protocol of arbitrary choice by using the examination protocol selection button 612 will be described.

In response to the operator touching the examination protocol selection button 612, the output control unit 303 displays an examination protocol selection screen 710 illustrated in FIG. 7 on the display unit 310. Each examination protocol includes a protocol name and information that indicates whether images are to be captured successively or one by one through examinations included in this examination protocol (in FIG. 7, “Auto”, “Manual”). Each examination protocol further includes information that indicates whether both-eyes imaging or single-eye imaging is to be performed (in FIG. 7, “Both”, “Single”), and shows the sequential order of imaging through the examinations. The sequential order of imaging through the examinations is assumed to be from the left to the right in FIG. 7. The sequential order thereof may be from the right to the left. They constitute an example of information that represents a plurality of examinations. The information that represents a plurality of examinations may be any kind of information as long as it shows the sequential order of the plurality of examinations in a recognizable manner. Examples of the examinations include fundus imaging, anterior eye segment imaging, fluorescent fundus imaging, fundus OCT imaging, anterior eye segment OCT imaging, and the like. In OCT imaging, a scanning pattern is further displayed. Examples of a scanning pattern corresponding to an examination regarding OCT imaging include wide scan, 3D scan, radial scan, cross scan, multi-cross scan, circle scan, raster scan, line scan, and the like, and the position of the fixation lamp is set for each of them. In the example illustrated in FIG. 7, the scanning patterns are represented by icons. A plurality of examination protocols is displayed on the examination protocol selection screen 710. One of them is Protocol 2 denoted as 711. Protocol 2 is set as an examination protocol according to which a wide scan image, a cross scan image, and a fundus image are captured successively. An examination protocol can be set arbitrarily on the initial setting screen 371, and the sequential order of examinations and the combination thereof can be selected freely. The protocol name set for the examinations can be changed, and any name (for example, Macula, Glaucoma, OCTA, etc.) can be set instead of Protocol 2.

Examinations with the same scanning pattern but with different angles of view or under other different imaging conditions may be included in different examination protocols respectively. Examples of a plurality of different examination protocols stored in the storage unit 302 include examination protocols according to which OCT 3D scan/multi-cross scan imaging and fundus imaging are performed, examination protocols according to which OCTA imaging with a plurality of different angles of view and fundus imaging are performed, etc. An imaging angle of view may be displayed near the icon, though not illustrated. For example, a field-of-view size of 13×10 may be displayed over the icon of wide scan included in Protocol 2 denoted as 711, a field-of-view size of 10×10 may be displayed over the icon of cross scan included therein, and an imaging angle of view of 45° may be displayed over the icon of the fundus image included therein. Icon design may be changed depending on the position (the center of the macula, the center of the posterior part, the center of the optic disc) of the fixation light. The position of the fixation light may be spelled out.

In the example illustrated in FIG. 7, eight types of examination protocol and a follow-up examination protocol 712 are displayed; however, this does not imply any limitation. The number of types of examination protocol may be three, or ten. However, it is recommendable to predefine the maximum display number because, firstly, display on the screen is impossible in a case where there are too many examination protocols and, secondly, it takes time to find a desired protocol in this case. The follow-up examination protocol 712 is an examination protocol that is not displayed when the subject is a new examinee but displayed when a follow-up observation is performed. As the examination protocol for follow-up use, this examination protocol is set automatically in accordance with past examination patterns of the subject who is the target of imaging. In the follow-up examination protocol 712, all scanning patterns included in past examination protocols may be displayed, or the examination protocol that was used in the immediately preceding imaging only may be displayed. The follow-up examination protocol 712 according to the present embodiment shows an example of displaying all scanning patterns included in past examination protocols. In a case where there are many scanning patterns, the display may be performed with different icon design, or with reduced icon size. That is, the control unit 300 suffices to control a display unit such that information that represents a plurality of examinations included in each of at least two examination protocols among a plurality of examination protocols stored is displayed on a protocol-by-protocol basis for the at least two examination protocols.

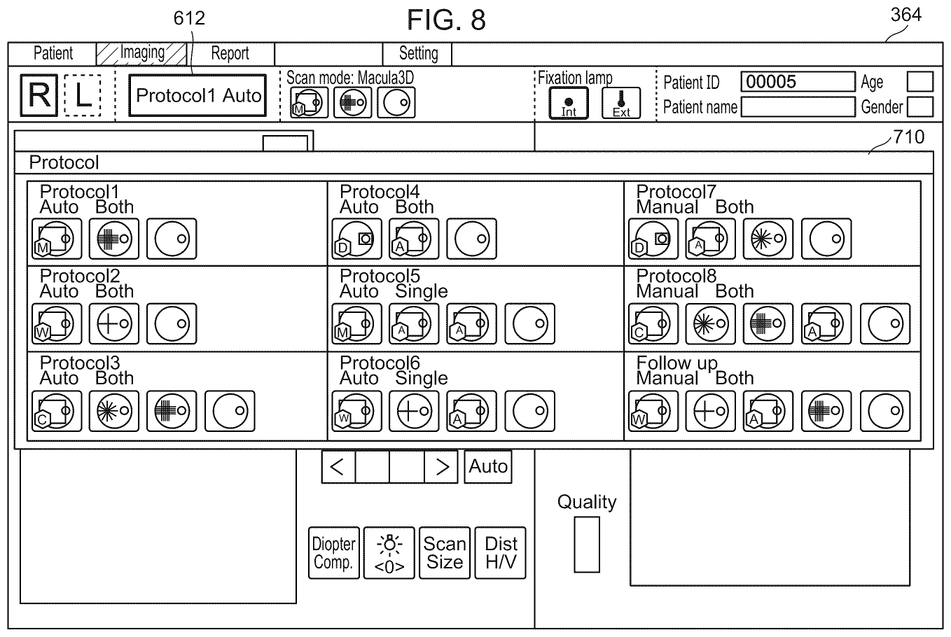

FIG. 8 illustrates a display example of the examination protocol selection screen 710 displayed together with the imaging screen 364 at the same time. For examination protocol determination, the operator performs a touch operation on the examination protocol selection screen 710 to select any examination protocol and, upon this determination, the output control unit 303 closes the examination protocol selection screen 710. Though the operation doubles as an operation for selecting the examination protocol and an operation for the examination protocol determination in the present embodiment, this does not imply any limitation. A non-illustrated OK button may be selected for the examination protocol determination. The present embodiment will be described using an example of a case where Protocol 2 is selected and, among OCT scanning patterns, wide scan and cross scan are executed, followed by fundus imaging.

Next, in step S103, a chin rest adjustment is performed using the chin rest adjuster 622. The relative Y position of the subject and the optical head unit 100 is adjusted by controlling the chin rest adjuster 622. The controlling of the chin rest adjuster 622 is not limited to operation by the operator. The control unit 300 may control it automatically. In a case where the control unit 300 performs automatic chin rest control, it is recommendable to perform this control before the subject puts his/her face on the chin rest, not in step S103. For example, since the face size of an adult is different from that of a child, a chin rest adjustment is performed depending on the subject. Therefore, the chin rest is sometimes moved up from a lower position for the next subject (movement in the opposite direction, downward from an upper position, could also happen). If an amount of movement of the chin rest adjuster 622 needed due to a subject change is large, it takes time for making a pre-imaging adjustment; therefore, the chin rest may be returned to its default position at the timing of a transition from the imaging screen 364 to the patient screen 362 after completion of image capturing the subject. The default position of the chin rest may be a single position. Alternatively, the default position may be based on the race, age, and gender read from the patient information that was entered on the patient screen, and the default position may differ from subject to subject.

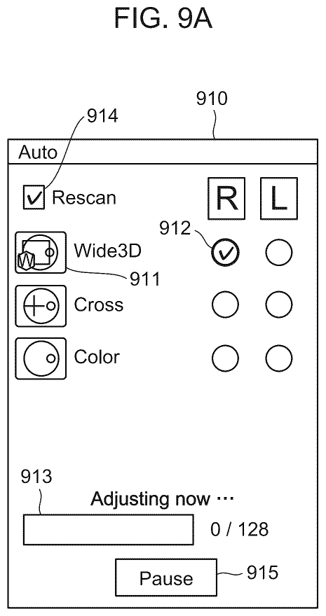

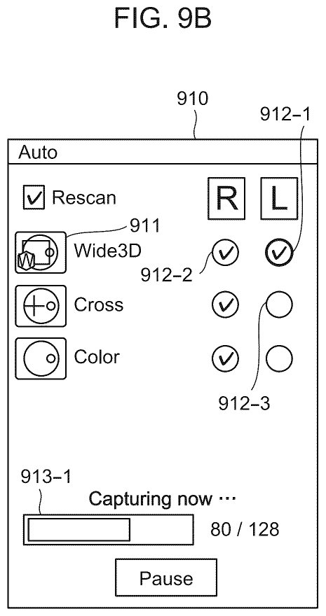

Next, in step S104, in response to the operator touching the start button 624, the image acquisition unit 304 starts acquiring a fundus image and acquiring an OCT tomographic image, and starts a series of examinations on the basis of the examination protocol having been set in step S102. The imaging control unit 301 performs an alignment of the apparatus and performs successive capturing of images of both eyes. When a plurality of images is captured successively in the present embodiment, a progress dialogue 910 illustrated in FIGS. 9A and 9B is displayed. FIG. 9A illustrates the progress of examinations that are under imaging now. Numeral 911 denotes names and icons as information that represents an examination protocol. Icons denoted as 912 represent “under capturing”, “capturing done”, and “waiting for capturing” as progress status. The icons 912 representing the progress status constitute an example of information that shows status regarding the plurality of examinations when the plurality of examinations included in the selected one examination protocol is executed successively. The information that shows status regarding the plurality of examinations may be, for example, information that shows the current examination progress status and represents any one of “capturing done”, “under capturing”, and “waiting for capturing” in the plurality of examinations included in the selected one examination protocol. Numeral 913 denotes a current status indicator, and indicates status such as “adjusting now”, “capturing now”, “changing eye L/R now”, or the like.

The relationship between 912 and 913 is as follows: 913 indicates the status (“adjusting now”, “capturing now”, etc.) of the examination that is indicated by 912 as “under capturing”. An example of a change in status is illustrated in FIG. 9B. In FIG. 9B, numeral 912-1 denotes “under capturing”, numeral 912-2 denotes “capturing done”, and numeral 912-3 denotes “waiting for capturing”. Though not illustrated, in a case where single-eye imaging in an Auto mode is performed, one column of the right eye only or the left eye only may be displayed; alternatively, though both columns are displayed, the progress status icons 912 for the eye that is not captured may be, for example, grayed out so that the operator knows that capturing is not performed for this gray-out-indicated eye. Upon completion of capturing an image for an examination, automatic image judgment processing may be performed, and “image OK” or “image not OK” may be displayed in addition to “capturing done”. For example, different colors may be used for display so that “OK” and “not OK” can be distinguished from each other though the type of the capturing-done icon is the same, or different icons may be assigned to “OK” and “not OK”. As an example of judging an image automatically by the image processing unit 305, for example, in a case of an OCT tomographic image, the level of image brightness may be used for the judgment because an empty image with no captured content is dark. In a case of a fundus image, a coal-black image and a snow-white image can be judged as “not OK”, or a flare at the periphery may be detected for a non-OK judgment. That is, it is possible to calculate an examination quality score on the basis of the luminance (maximum value, minimum value, median value, average value, variance, standard deviation, histogram) of an image included in the examination, image contrast, image edge, etc., and it is possible to perform labeling of “not OK” in a case where the quality score of the examination is equal to or lower than a reference value. The reference value of the quality score may differ from patient to patient. For example, it is possible to acquire an average value or a median value of acquired quality scores of a plurality of examinations, and it is possible to label an examination whose quality score is extremely low in comparison with this value as “not OK”. By this means, it is possible to properly label the examination as “OK” or “not OK” even for a patient whose quality score is low on average and even for a patient whose quality score is high on average. A machine learning engine having been trained with normal images and abnormal images may be used for making an abnormal image judgment, or a machine learning engine having been trained with scored images (scored with successive numerical values such as a score of 100 points for a highest grade image and a score of 0 point for a lowest grade image) may be used for making a score-based judgment. The machine learning engine includes an algorithm regarding deep learning such as a convolutional neural network (CNN). During-imaging information such as eye stability during imaging, the number of times of rescanning during imaging, or the like may be used for labeling the examination as “OK” or “not OK”.

An indicator 913-1 indicates that an image is being captured now. That is, FIG. 9A is an example that illustrates a state in which the apparatus is being adjusted now for the purpose of preparing for capturing a wide scan image of the right eye, and FIG. 9B is an example that illustrates a state in which, after the completion of the right-eye capturing, a wide scan image of the left eye is being captured now.

Numeral 914 denotes a checkbox for switching Rescan ON/OFF. In a case where rescanning is executed many times during OCT imaging because of visual fixation or an eye state of the subject and thus it takes time for imaging, this checkbox can be used for giving a rescanning cancel instruction by the operator before the end of the imaging. When the rescanning cancel instruction is given, the imaging control unit 301 performs imaging without rescanning. Numeral 915 denotes a button for giving an instruction for pausing an examination protocol according to which images are captured successively. When the operator wants to suspend the process because the examinee is tired or wants to manually adjust the positional relationship between the apparatus and his/her eye, this button can be used for pausing the successive capturing.

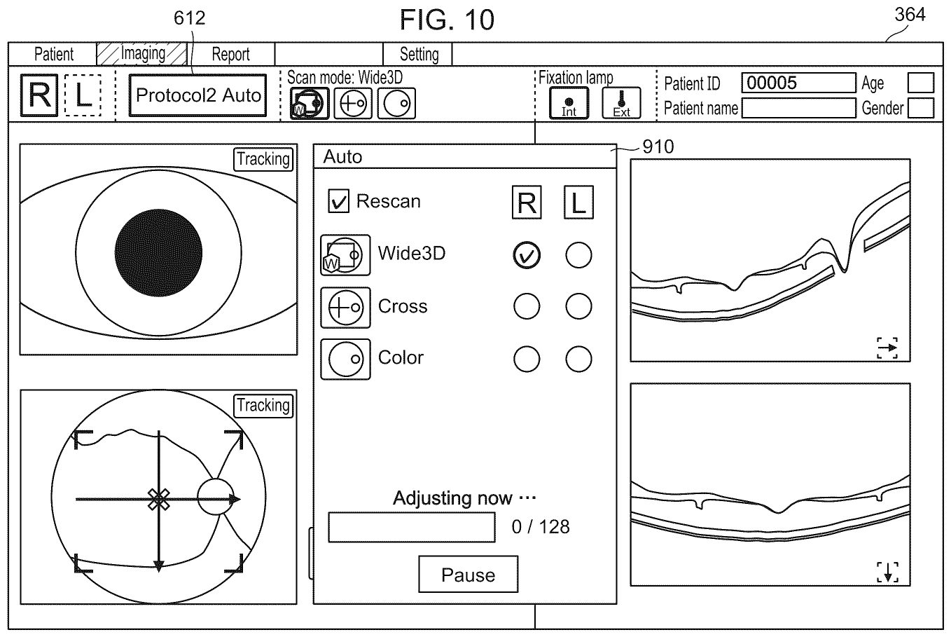

FIG. 10 illustrates a display example of the progress dialogue 910 displayed together with the imaging screen 364 at the same time. In a case of an examination protocol according to which images are captured successively, in response to the operator touching the start button 624, the output control unit 303 displays the progress dialogue 910. Then, alignment and imaging are performed automatically in a successive manner, with the progress dialogue 910 displayed.

Next, in step S105, the imaging control unit 301 performs various kinds of alignment adjustment. Since images are acquired when various kinds of alignment adjustment are performed, as illustrated in FIG. 10, after the start of the examination protocol in step S104, a fundus image is displayed in the fundus observation image display section 630, and tomographic images are displayed in the OCT tomographic image display section 650.

The imaging control unit 301 instructs the head driving unit 170 to reduce an amount of positional deviation calculated by the image processing unit 305. By driving the three non-illustrated motors, the head driving unit 170 causes the optical head unit 100 to move for a positional adjustment with respect to the subject's eye E in three-dimensional directions (X, Y, and Z). After the completion of the alignment adjustment, for each image capturing, the final alignment position is stored into the storage unit 302.

Next, a focus adjustment is performed. The image processing unit 305 acquires a fundus image, and calculates the contrast of the acquired fundus image. The imaging control unit 301 causes the focus lens 133 to move in such a way as to increase the contrast of the fundus image.

A coherence gate adjustment is further performed. The image processing unit 305 acquires an OCT image while moving the coherence gate adjuster, and detects the brightness of the tomographic image. The imaging control unit 301 drives the mirror 160 to a position where the brightness of the tomographic image becomes large to adjust the optical path length of reference light.

The alignment processes in step S105 may be performed in a different sequential order, or may be performed concurrently. For example, a fine alignment adjustment and a focus adjustment may be started at the same time upon the completion of a rough alignment adjustment. After the completion of alignment operation of each of an alignment adjustment, a focus adjustment, and a coherence gate adjustment, alignment operation of each of an alignment adjustment, a focus adjustment, and a coherence gate adjustment may be performed again as fine adjustments.

Upon the imaging control unit 301 completing the automatic alignment adjustment, in step S106, the imaging control unit 301 scans the OCTX scanner 153-1 and the OCTY scanner 153-2 to perform image capturing. In the image capturing in step S106, a tracking is performed for each of a fundus image and an anterior eye segment image. First, an example of the tracking of a fundus image will now be described. Immediately before starting the capturing of a tomographic image by using the OCT optical system, the control unit 300 controls the imaging optical system 100 to cause the image acquisition unit 304 to acquire a frontal fundus image and cause the storage unit 302 to store the acquired frontal fundus image as a tracking-use reference image. Then, the capturing of a tomographic image is started, and subsequent processing is performed in parallel while performing B scan and C scan by the OCT optical system.

The control unit 300 controls the imaging optical system 100 to generate a tracking-use target image (frontal fundus image). Then, the control unit 300 sends the tracking-use target image, together with the tracking-use reference image stored in the storage unit 302, to the imaging control unit 301. The image processing unit 305 calculates the positional deviation of the tracking-use target image from the tracking-use reference image to acquire an amount of movement of the fundus of the subject's eye E that has occurred during the time between acquiring these images. This movement of the fundus occurs due to the known motion of the subject's eye such as, for example, involuntary eye movement or the motion of the subject. After acquiring the amount of movement, the imaging control unit 301 performs control to correct the position of irradiation with measurement light by the OCTX scanner 153-1 and the OCTY scanner 153-2 on the basis of the acquired amount of movement of the fundus of the subject's eye E.

Next, the tracking of an anterior eye segment image will now be described. The image processing unit 305 detects the position of the center of gravity of a pupil area by applying binary processing that is based on a predetermined threshold to the pupil area in an anterior eye segment image. The predetermined threshold may be a fixed threshold, or a dynamic threshold as in a discriminant analysis. An amount of positional deviation in the X, Y direction is calculated on the basis of a difference between the calculated position of the center of gravity of the pupil area and a predetermined position of the anterior eye segment image, and the imaging control unit 301 controls the position (X, Y, Z) of the optical head unit 100.

In step S107, it is determined whether the imaging has finished for the examinations set in the examination protocol or not. In the present embodiment, it is determined whether wide scan, cross scan, and fundus imaging have finished for one eye or not. The process proceeds to step S108 in a case where the capturing of images of them has not finished yet. The process proceeds to step S109 in a case where the capturing of images of them has finished.

In step S108, it is determined whether the alignment in step S105 is necessary or not. In an examination protocol according to which images are captured successively through different examinations, there is no need to perform an alignment again if the state of the eye of the subject is stable when successive imaging for examinations with the same position of the fixation light is performed. Or even with a different position of the fixation light, the amount of eye movement is small for a change between the center of the macula and the center of the posterior part and, therefore, continuing the performing of the tracking makes the alignment unnecessary.

On the other hand, in a case where a large eye movement is involved as in a change in the position of the fixation light between the center of the macula and the center of the optic disc, an alignment is performed again. In a tracking using a reference image, reference image acquisition is redone. Factors as to whether to perform the alignment or not are not limited to a change in the position of the fixation light; the image processing unit 305 may determine whether to perform the alignment or not by judging a state of the eye of the subject by using any one of an anterior eye segment observation image, a fundus observation image, and an OCT image.

In comparison with OCT imaging, an alignment with higher precision is required for fundus imaging because of a flare. Therefore, an alignment may be performed at the time of switching from OCT imaging to fundus imaging in an examination protocol. Alternatively, image capturing may be continued just with a tracking, by changing the threshold of the tracking to higher-precision one at the time of switching from OCT imaging to fundus imaging.

Though both an alignment and a tracking are performed for an anterior eye segment observation image, it is possible to change the parameters of control (threshold, search range) and the method of control. For example, for an alignment, a search range is inevitably wide because detection is started from a state in which an initial eye position is unknown, whereas the search range may be made narrower for a tracking because its target is the already detected pupil. With regard to control, the position of the optical head unit 100 may be controlled in three dimensions (X, Y, Z) when an alignment is performed, whereas the position of the optical head unit 100 may be controlled in two dimensions (X, Y) when a tracking is performed. In this case, the tracking in the Z direction during imaging may be realized by performing a coherence gate adjustment,

In a case where it is determined in step S108 that an alignment is necessary, the image capturing in step S106 is performed after performing the above-described alignment in step S105. In a case where it is determined in step S108 that an alignment is unnecessary, the image capturing in step S106 is performed.

The above-described processing from step S105 to step S108 regarding image capturing is performed for the one eye a plurality of number of times corresponding to the number of the examinations included in the examination protocol.

In step S109, the control unit 300 determines whether to perform switching between the left eye and the right eye or not. In a case where the examination protocol that was set in step S102 is an examination protocol according to which imaging is performed for both eyes and where the processing from step S105 to step S108 has finished for one eye only, the process proceeds to step S110. In step S110, the imaging control unit 301 causes the optical head unit 100 to move laterally. After the lateral movement, the above-described processing from step S105 to step S108 is repeated.

In a case where the set examination protocol is an examination protocol according to which imaging is performed for one eye, or where the set examination protocol is an examination protocol according to which imaging is performed for both eyes but the processing has finished for both eyes, the process proceeds to step S111.

In step S111, all of the examinations for which imaging has been done in the examination protocol are displayed for confirmation. As a state of this display of the results, the images included in all of the examinations may be displayed in a switched manner one by one on an examination-by-examination basis, or the results of the plurality of examinations may be displayed together in a single-view format. For example, in a case of the examination protocol according to the present embodiment, which includes wide scan, cross scan, and fundus imaging, the examination results of them for both eyes may be arranged and displayed in a single-view format. The results of performing automatic image judgment processing by the image processing unit 305 (“OK” or “not OK”) may be further displayed.

In step S112, the operator determines whether the examination protocol has completed or not; after confirming the results of all of the examinations included in the examination protocol, if the operator determines that the examination protocol has completed and gives a completion instruction on the confirmation screen, the series of the examinations ends. On the other hand, if there is any examination for which the operator who has confirmed the results in step S111 determines that there is a need to perform image capturing again, image capturing is performed again for this examination. An instruction for performing image capturing again can be given not only for one of the plurality of examinations included in the examination protocol alone but also for a plurality of examinations together. For example, in the examination protocol according to the present embodiment, which includes wide scan, cross scan, and fundus imaging for both eyes, an instruction for performing image capturing again is given for three examinations in total of right-eye wide scan, right-eye cross scan, and left-eye fundus imaging. To perform image capturing for these examinations successively, flag information or the like is set to designate that image capturing should be performed for these examinations only among those in the examination protocol. By this means, the image capturing is performed again for the designated examinations only.

After the completion of performing image capturing, in the result display in step S111 again, the images having been captured again for the designated examinations are displayed together with display that lets the operator know that they are the images having been captured again.

The operator confirms the results again and determines again whether all of the examination have completed or not. It is possible to give an instruction for performing image capturing again also for the captured-again result(s). Moreover, at this time, it is possible to give an instruction for additionally performing image capturing again also for an examination(s) for which a capturing-again instruction was not given first.

As described in the present embodiment above, when an operator is to make a choice from among a plurality of examination protocols, examination conditions included in the examination protocols are presented, and this makes it possible for the operator to select one examination protocol from among the plurality of examination protocols easily and to cause the selected one to be executed.

Furthermore, since information about the progress status of the examination protocol is presented to the operator, the operator can know the progress status of the examinations. Therefore, the operator can operate a plurality of apparatuses simultaneously, and, even if the operator leaves the apparatus unattended temporarily, the operator can know the progress of the examinations upon coming back.

### Second Embodiment

In the present embodiment, an ophthalmic apparatus that pauses an examination flow at a timing of arbitrary choice will be described; specifically, the ophthalmic apparatus according to the present embodiment makes it possible to pause an examination flow at a timing of arbitrary choice while confirming the progress status of examinations in an examination protocol involving examination execution multiple times. When a plurality of examinations is executed automatically in a successive manner, a failure in image capturing could happen depending on a degree of fatigue of the subject, due to drying of the subject's eye, due to an automatic alignment error, or due to any other problem. In view of this, the present embodiment aims to carry out successive examinations efficiently and obtain stable examination results. The configuration of an apparatus according to the second embodiment, the configuration of a control unit according thereto, a method of detecting relative position information according thereto, and a method of performing adjustment operation according thereto are the same as the configuration of an apparatus according to the first embodiment, the configuration of a control unit according thereto, a method of detecting relative position information according thereto, and a method of performing adjustment operation according thereto; therefore, an explanation of them is omitted. The operation flow according to the present embodiment may be, at least partially, executed in combination with, or executed with replacement by, the operation flow, etc. according to any of the above-described various examples of the foregoing embodiment, unless such a combination or such a replacement is contradictory.

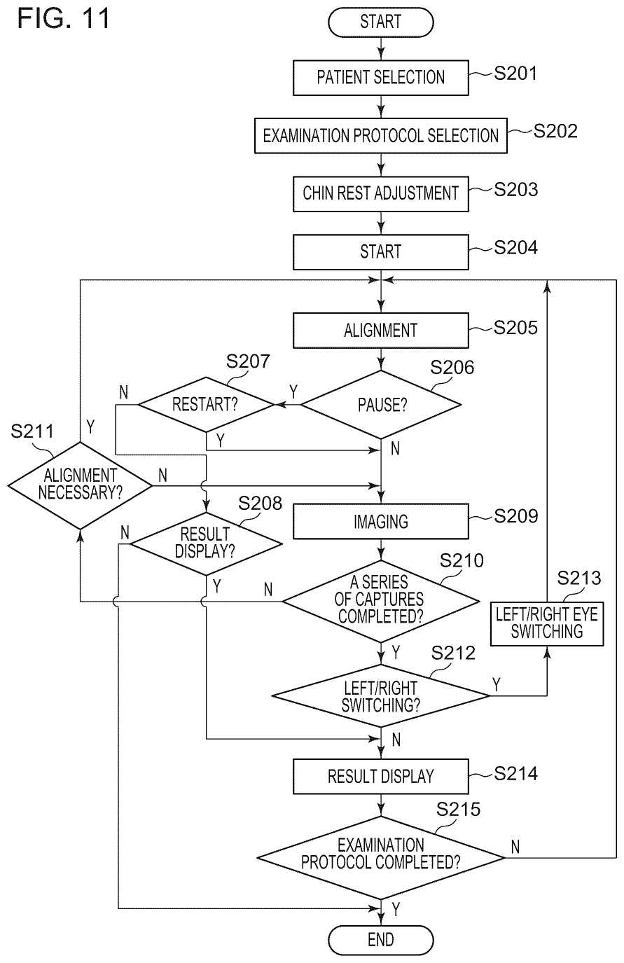

With reference to FIGS. 11 to 13, the operation flow of examinations according to the present embodiment will now be described.

FIG. 11 is an examination flowchart according to the present embodiment. The flow from the patient selection in step S201 to the alignment in step S205 is the same as the flow from the patient selection in step S101 to the alignment in step S105 that has been described in the first embodiment. The flow from the image capturing in step S209 to the switching between the left eye and the right eye in step S213 is the same as the flow from the image capturing in step S106 to the switching between the left eye and the right eye in step S110 that has been described in the first embodiment. Therefore, an explanation of these same parts of the flow is omitted.

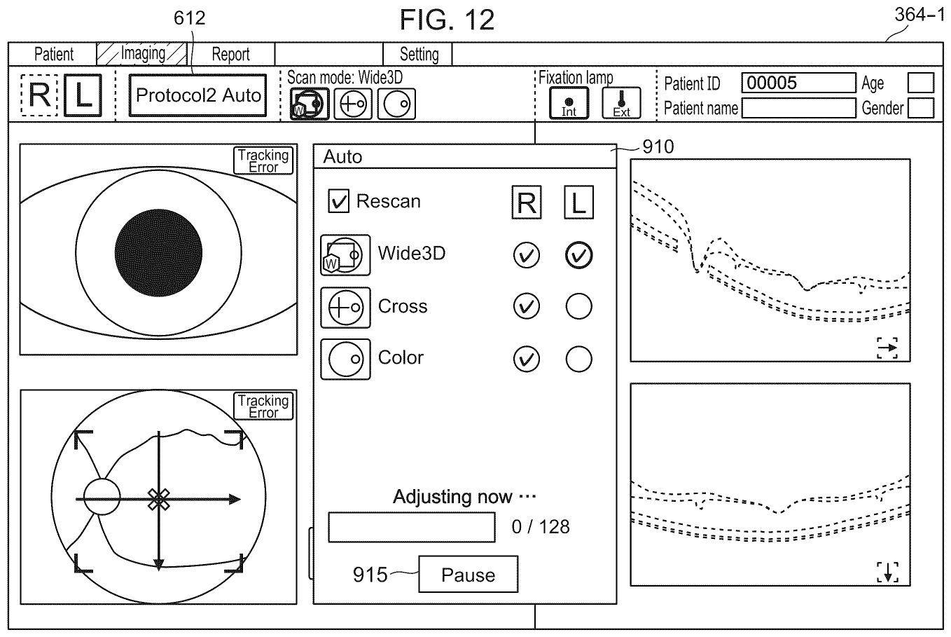

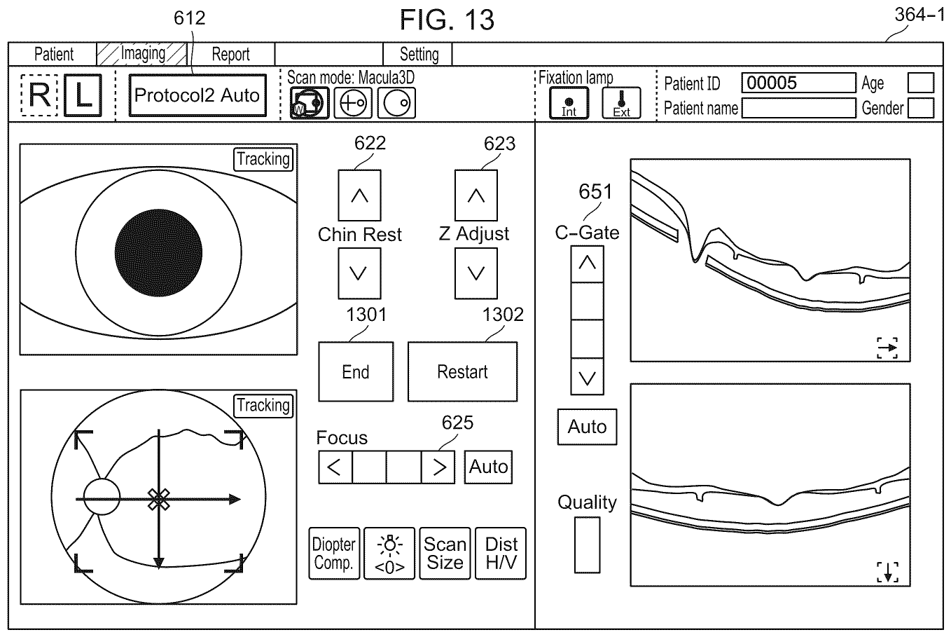

In step S206, the operator gives a pause instruction. This will now be described with reference to FIGS. 12 and 13. Taking a look at the progress status of the examinations and a state of the eye of the subject, the operator determines whether to execute a pause or not. For example, FIG. 12 illustrates an example of giving a pause instruction because of a poor alignment or a poor eye state while an automatic pre-imaging alignment for the left eye is being performed after the completion of capturing images for the right eye. The operator touches the pause button 915 in the progress dialogue 910 illustrated in FIG. 12. In response to the touch on the pause button 915, the output control unit 303 closes the progress dialogue 910, and displays an imaging screen 364-1 illustrated in FIG. 13. The imaging screen 364-1 disclosed in the second embodiment is basically the same screen as the imaging screen 364 disclosed in the first embodiment but is different therefrom in that it includes an end button 1301 and a restart button 1302, in place of the start button 624. Since the examination protocol has already been started by touching the start button 624, the restart button 1302, which is needed for a restart after the pause, and the end button 1301, are displayed instead here. In the present embodiment, button display is changed in accordance with the operation that is being performed. However, this does not imply any limitation. All of the start button 624, the end button 1301, and the restart button 1302 may be displayed, and the selection of necessary buttons only may be enabled by graying out an unnecessary button, for example.

In step S207, the operator performs various kinds of adjustment manually on the imaging screen 364-1 illustrated in FIG. 13. For example, the adjustments are performed using the chin rest adjuster 622, the Z adjuster 623, the focus adjuster 625, and the coherence gate adjuster 651 respectively. In a case where, as the result of these adjustments, the operator wants the examination to be restarted, the operator touches the restart button 1302. In response to this touch, the output control unit 303 displays the progress dialogue 910, and restarts the imaging in the examination protocol.

In a case where it is difficult to perform the automatic imaging successively although the operator has performed various adjustments manually, the operator can abort the successive capturing of images in the examination protocol.

In response to the operator touching the end button 1301, the process proceeds to step S208.

In step S208, a non-illustrated confirmation dialogue as to whether or not to display the results of examinations having been done halfway is displayed. In a case where the operator choose not to confirm the results, the examination protocol according to which images are captured successively is terminated without displaying the results.

In a case where the operator choose to confirm the results, the process proceeds to step S214, and the results are displayed.

In step S214, the results are displayed. In the result display of a plurality of examinations, the results of examinations for which image capturing has completed are displayed in a single-view format. Alternatively, the results may be displayed one by one for the respective examinations. The result display may be performed such that the operator can know which one or more of the examinations that were supposed to be executed in the examination protocol is/are in a capturing-not-completed state. On the result display screen, the operator is able to give an instruction for performing image capturing again. Image capturing can be performed again for a capturing-done examination(s) and for a yet-to-be-done examination(s).

In the present embodiment, it has been described that a pause is executed between the alignment is step S205 and the image capturing in step S209; however, this does not imply any limitation. A pause operation may be performed during image capturing. In such a case, ongoing image capturing may be paused, and, after various adjustments are made manually, the continuation of the paused image capturing may be executed. Specifically, in a case where 128 shots of B scan are to be taken, image capturing may be paused upon completion of 30 shots, and, after adjustments, the paused image capturing may be resumed from the 31st shot. Alternatively, in the restart after various adjustments are made manually after the pause, the image capturing may be redone from the beginning. Specifically, in a case where 128 shots of B scan are to be taken, image capturing may be paused upon completion of 30 shots, and, after adjustments, the image capturing may be performed all over again from the first shot.

As described in the present embodiment above, the operator can give a pause instruction before the completion while seeing information about the progress status of the examination protocol, and can give a restart instruction after the pause. This makes it possible to carry out successive examinations efficiently and obtain stable examination results.

### Third Embodiment

In the present embodiment, an ophthalmic apparatus that selectively executes an examination(s) of arbitrary choice in an examination protocol according to which examinations are executed manually will be described. The configuration of an apparatus according to the third embodiment, the configuration of a control unit according thereto, a method of detecting relative position information according thereto, and a method of performing adjustment operation according thereto are the same as the configuration of an apparatus according to the first embodiment and the second embodiment, the configuration of a control unit according thereto, a method of detecting relative position information according thereto, and a method of performing adjustment operation according thereto; therefore, an explanation of them is omitted. The operation flow according to the present embodiment may be, at least partially, executed in combination with, or executed with replacement by, the operation flow, etc. according to any of the above-described various examples of the foregoing embodiments, unless such a combination or such a replacement is contradictory.

With reference to FIGS. 7 and 14 to 18, the operation flow of examinations according to the present embodiment will now be described. Examinations according to the present embodiment will be described using an example of a case where Protocol 8 is selected on the examination protocol selection screen 710 according to the first embodiment and where OCTA imaging is performed for the right eye and fundus imaging is performed for the left eye.

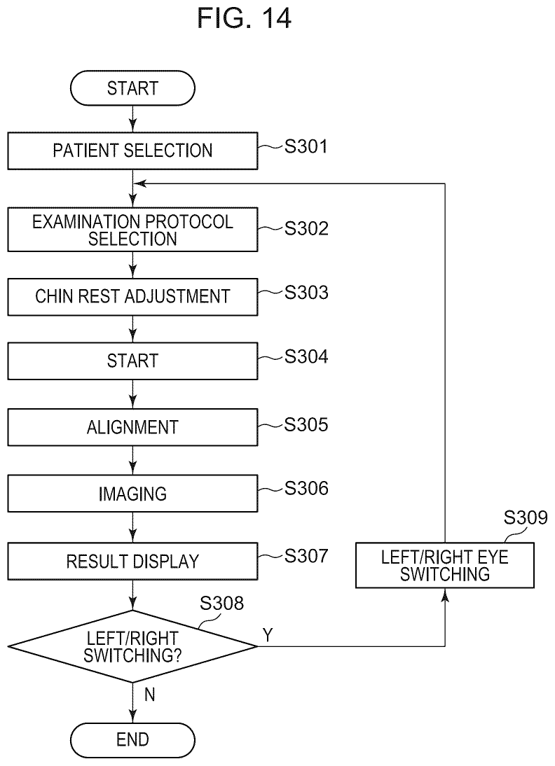

FIG. 14 is an examination flowchart according to the present embodiment. Since the patient selection in step S301 is the same as the patient selection in step S101, an explanation of it is omitted.

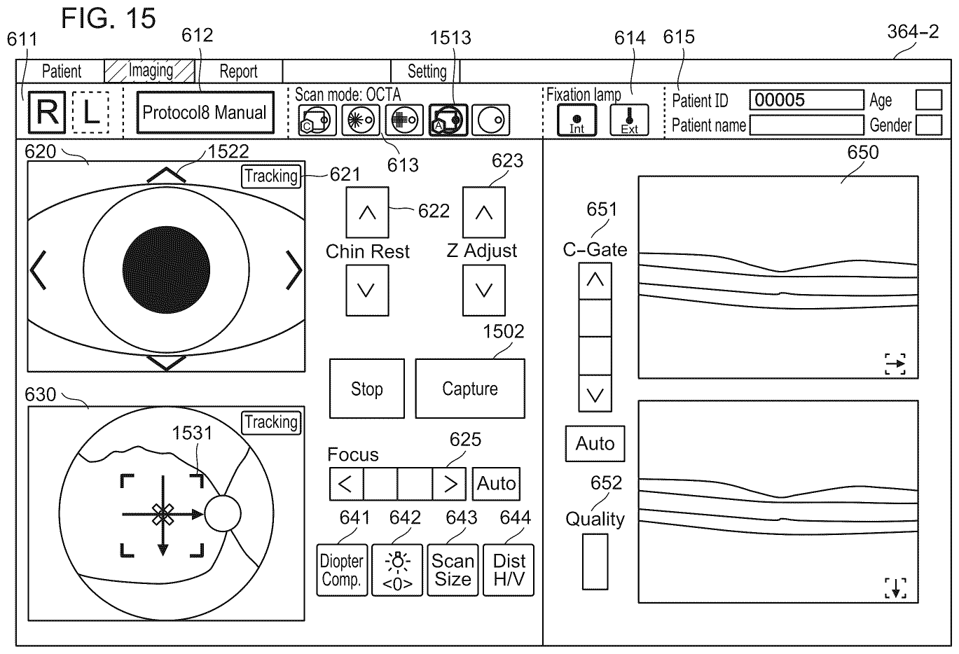

In step S302, Protocol 8 is selected on the examination protocol selection screen 710 illustrated in FIG. 7. Then, on an imaging screen 364-2 illustrated in FIG. 15, an icon 1513 for OCTA imaging is touched selectively from among the icons 613 of examinations included in the examination protocol.

In step S303, the position of the chin rest is adjusted in the same manner as done in step S103.

In step S304, the operator operates the start button on the imaging screen to give a start instruction. In response to the start instruction, the image acquisition unit 304 starts acquiring a fundus image to be displayed in the fundus observation image display section 630, and tomographic images to be displayed in the OCT tomographic image display section 650.

In step S305, the imaging control unit 301 performs various kinds of alignment adjustment automatically in the same manner as done in step S105. As various kinds of adjustment, the optical head unit 100 is moved in three-dimensional directions (X, Y, Z) with respect to the subject's eye E, and a focus adjustment and a coherence gate adjustment are performed. After the completion of various kinds of alignment adjustment by the imaging control unit 301, the operator can perform manual adjustments.

For example, an optical head control button 1522 for changing the position (X, Y) of the optical head unit 100 with respect to the subject's eye E may be displayed in the anterior eye segment observation image display section 620 so that fine adjustments can be made. The operator may directly touch the anterior eye segment image displayed in the anterior eye segment observation image display section 620 and control the optical head unit 100 such that the touched position comes to the center, or may touch the optical head control button 1522 to make fine adjustments. It is recommendable that the optical head control button 1522 be used for fine adjustments by finely controlling the optical head unit 100 when operated. Besides this adjustment, the operator may control the Z position of the optical head unit 100 by touching the Z adjuster 623, may perform focus control by touching the focus adjuster 625, and may make a coherence gate adjustment by touching the coherence gate adjuster 651. In addition to various alignment adjustments, an OCT (OCTA) scan position change and/or an OCT (OCTA) scan size change may be made. For example, in FIG. 15, a mark 1531 indicating a scan position and a scan range and superimposed on the fundus observation image displayed in the fundus observation image display section 630 may be dragged for making a position change, or any of four corners of this mark may be operated for making a size change. Alternatively, a non-illustrated size adjuster may be displayed in response to touching the OCT scan size adjustment button 643, and the scan size may be changed using the displayed adjuster.

In step S306, image capturing is performed in response to touching an imaging button 1502 on the imaging screen 364-2.

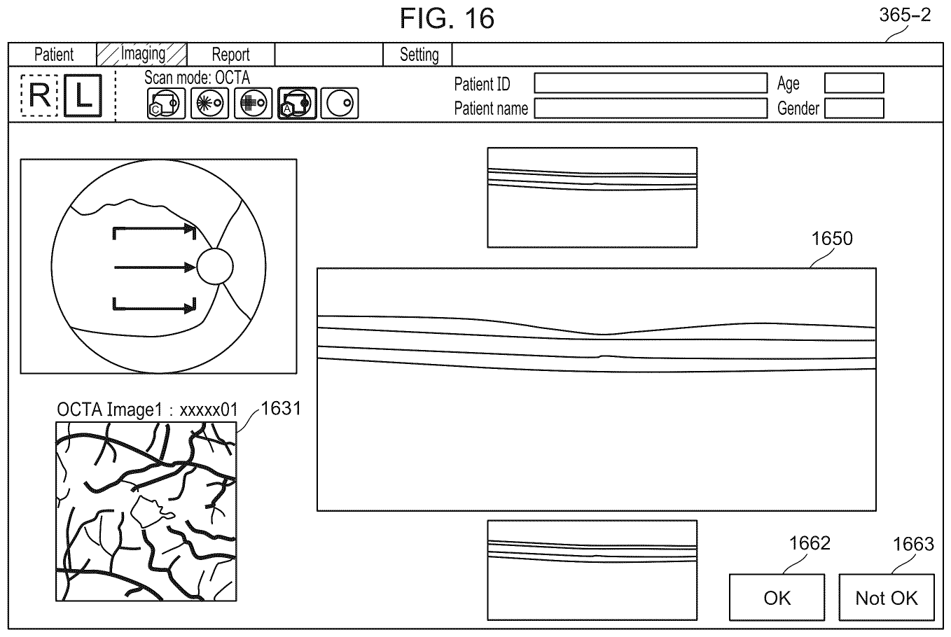

In step S307, the results of the image capturing are displayed. An example of an imaging result display screen 365-2 for displaying the imaging results of one examination is illustrated in FIG. 16. In a case of an OCTA examination, an OCTA image 1631 is displayed as the imaging results, besides a tomographic image 1650. If there is no problem in the imaging results, the operator touches an OK button 1662 to go on to the next examination. If the operator wants image capturing to be redone, the operator may touch a “not OK” button 1663 to instruct that image capturing be performed again.

If switching between the left eye and the right eye is determined to be necessary in step S308, the process proceeds to S309. The examination flow finishes if the instructed examination is a single-eye-only examination or if the instructed examination has completed for both eyes. Though an example of performing a single examination for one eye is described in the present embodiment, this does not imply any limitation. A plurality of examinations may be performed for one eye before switching between the left eye and the right eye is performed. In such a case, the process returns to the examination protocol selection in step S302, an examination icon of arbitrary choice is selected from among the examination icons 613, and the same flow as the above-described flow or similar thereto can be executed. The process that suffices to be executed just once for the subject, such as a chin rest adjustment, may be skipped. An adjustment may be performed again if needed.

In step S309, for the purpose of switching between the left eye and the right eye, the operator touches the L button of the left/right eye button 611 to move the optical head unit 100 from the right eye to the left eye.

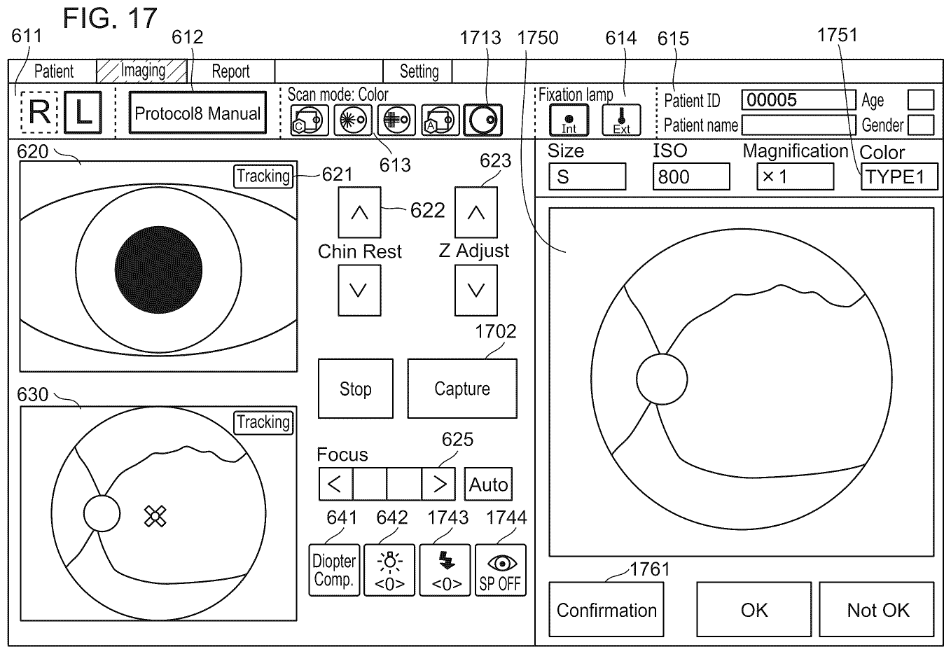

In step S302, fundus imaging of the left eye is performed. An example of an imaging screen for capturing a fundus image will now be described with reference to FIG. 17. The operator touches an icon 1713 for capturing a fundus image from among the icons 613 of examinations included in the examination protocol. In response to this touch, a transition from the OCT imaging screen to a screen for capturing a fundus image occurs. In FIG. 17, a light amount adjustment button 1743 and a small pupil mode setting button 1744, which are used for fundus imaging, are displayed in place of the OCT scan size adjustment button 643 and the OCT scan interval adjustment button 644, which are used for OCT imaging. Numeral 1751 denotes various fundus imaging parameters.

The fundus imaging parameters include, for example, fundus image size, ISO sensitivity, magnification, color setting, etc.

In step S304, the operator operates the start button on the imaging screen to give a start instruction. In response to the start instruction, the image acquisition unit 304 starts acquiring a fundus image to be displayed in the fundus observation image display section 630.

In step S305, for fundus imaging, the optical head unit 100 is moved in three-dimensional directions (X, Y, Z) with respect to the subject's eye E, and a focus adjustment is performed. Also in fundus imaging, similarly to OCT imaging, after the completion of automatic alignments by the imaging control unit 301, the operator can perform manual adjustments.

In step S306, in response to touching an imaging button 1702, the imaging control unit 301 performs fundus image capturing using visible light under imaging conditions defined in the examination protocol.

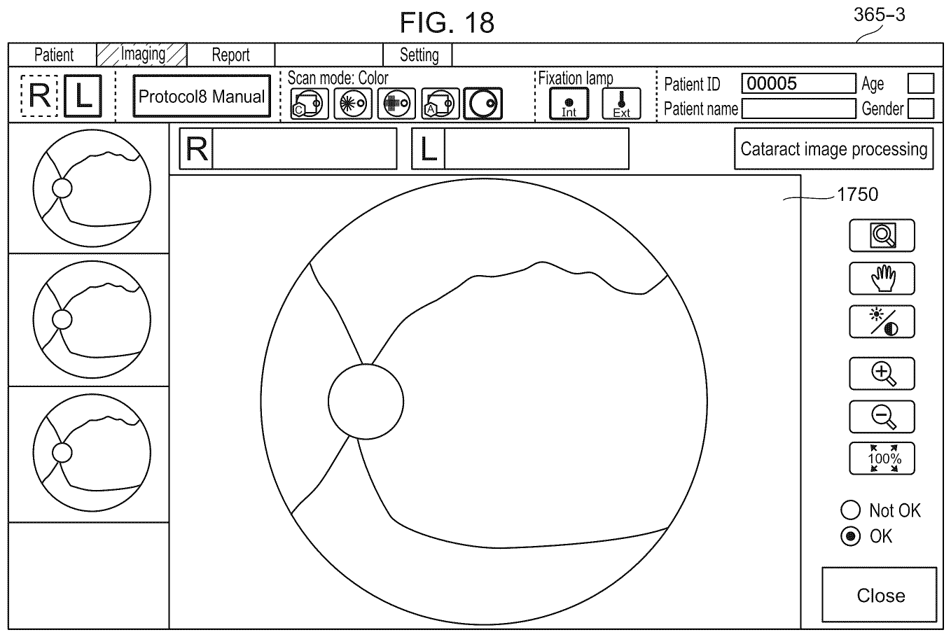

In step S307, a fundus image denoted as 1750 is displayed. In fundus image capturing, not only the image 1750 but also a fundus imaging result confirmation screen 365-3 illustrated in FIG. 18 may be displayed in response to the operator touching a confirmation button 1761 for the purpose of taking a closer look at the image. On the fundus imaging result confirmation screen 365-3 illustrated in FIG. 18, the operator can confirm images having been subjected to various kinds of image processing (image processing for a cataract, contrast enhancement processing), a red-free image (an image whose red components have been reduced by means of digital processing), and a cobalt image (an image whose blue components have been reduced by means of digital processing). Though another screen such as one illustrated in FIG. 18 is displayed in the example disclosed in the present embodiment, this does not imply any limitation. The display of the fundus image 1750 in FIG. 17 may be switched to a red-free image or a cobalt image.

Though a fundus image capturing screen has been described in the present embodiment, this screen is an imaging screen used also in a case where automatic successive capturing of images are performed through examinations included in an examination protocol in the first embodiment and the second embodiment. Switching between an OCT imaging screen and a fundus imaging screen is performed automatically in accordance with automatic switching between examinations.

As described in the present embodiment above, since a plurality of examinations included in an examination protocol is displayed, it is possible to execute an examination of arbitrary choice from among the plurality of examinations. This makes it possible to carry out the examination of arbitrary choice efficiently.

### Fourth Embodiment

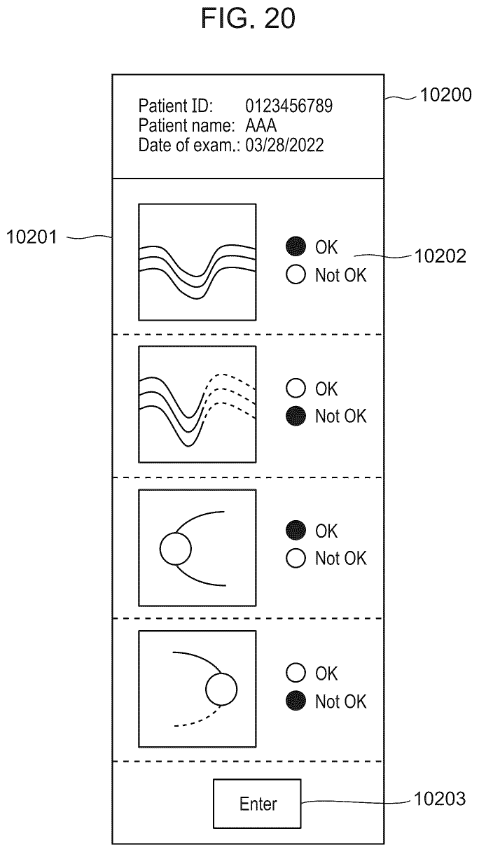

In the present embodiment, with reference to FIG. 20, a confirmation screen for confirming acquired images corresponding to a plurality of examinations in a batch will now be described. First, the control unit 300 controls the display unit 310 to display acquired images corresponding to a plurality of examinations 10201 in an arranged manner on a confirmation screen 10200. The control unit 300 may be built in an ophthalmic apparatus, or may be an example of an information processing apparatus provided separately from an ophthalmic apparatus and is connected to the ophthalmic apparatus such that communication can be performed therebetween. In addition, the control unit 300 controls the display unit 310 to display, on the confirmation screen 10200, for each examination 10201, a success/failure designation button (for example, OK/not OK button) 10202 for designating whether the acquired result of this examination is a success (OK) or a failure (not OK). In addition, the control unit 300 controls the display unit 310 to display, on the confirmation screen 10200, an Enter button 10203 for finalizing each designated success or failure. The person conducting the examination confirms the images having been acquired successively for the plurality of examinations 10201 (the results of the plurality of examinations included in the examination protocol), designates for each examination whether this examination is successful or not by operating the success/failure designation button 10202, and then finalizes the success or failure having been designated for each examination by operating the Enter button 10203. Upon the finalization of the successes/failures according to the instruction from the person conducting the examination via the Enter button 10203 on the confirmation screen 10200, the control unit 300 commands that different finalization processing be performed, depending on the success or failure of each examination. For example, as the finalization processing for each examination whose results have been designated as “success”, accompanying supplementary information “success” may be affixed to the results of the examination, the results of the examination may be stored into a local storage, report printing may be executed, or the results of the examination may be transferred to an external system. On the other hand, as the finalization processing for each examination whose results have been designated as “failure”, accompanying supplementary information “failure” may be affixed to the results of the examination, the results of the examination may be discarded without being stored, or the results of the examination may be configured not to be transferred to an external system. As described above, the processing performed at the time of finalizing the success/failure designation may vary depending on whether the examination is successful or not. In a case where there exists any examination whose results have not been designated as “success” nor as “failure”, the finalizing of this examination may be left pending, and the finalization processing for this examination may be performed at another timing of success/failure finalization. In the present embodiment, the confirmation screen 10200 is assumed to terminate simultaneously with the finalization processing via the Enter button 10203; instead, a non-illustrated button for closing the confirmation screen may be displayed besides the Enter button 10203.

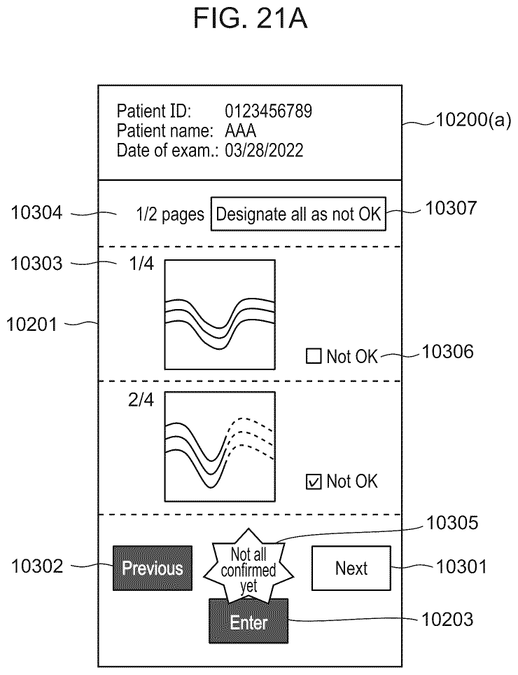

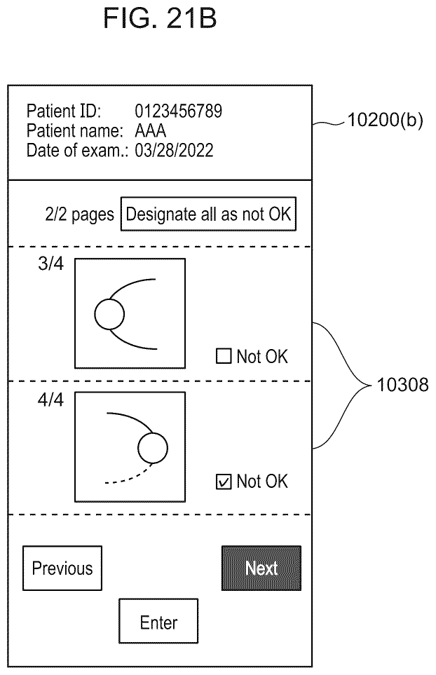

There is no need to necessarily display the acquired results of all of the plurality of examinations at one time in the confirmation screen 10200. For example, as illustrated in FIGS. 21A and 21B, switching between a confirmation screen 10200(a) and a confirmation screen 10200(b) may be made available using a Next button 10301 and a Previous button 10302 or the like so that the results of all of the plurality of examinations are viewable. For preventing the operator from forgetting to confirm the results of any examination, it will be effective to perform examination numbering display 10303 showing the total number of examinations and a serial number of each examination and to display page information 10304 showing the total number of pages of the confirmation screen 10200 and the current page number. The examination numbering display 10303 is an example of display that makes it possible to recognize the sequential order of a plurality of examinations having been executed in accordance with an examination protocol. The examination number 10303 is an example of information about the sequential order of an examination corresponding to each of the results of examinations that are displayed among the results of a plurality of examinations. In a case where there exists any examination that has never been displayed in the confirmation screen 10200, a symbol and/or characters 10305 may be displayed for letting the operator know that a yet-to-be-confirmed examination 10308 exists. In a case where a yet-to-be-confirmed examination 10308 exists, the Enter button 10203 may be disabled so that the success/failure having been designated for each examination cannot be finalized, or the confirmation screen 10200 may be configured to be unable to be terminated. In the present embodiment, an examination is deemed as “confirmation done” by being displayed on the confirmation screen 10200; however, a non-illustrated “Confirmation done” button may be associated with each examination, and the status of the examination may be configured not to become “Confirmation done” unless an explicit designation of “Confirmation done” by the operator. The means for switching examinations displayed on the confirmation screen 10200 is not limited to the Next button 10301 and the Previous button 10302. This switching may be realized by a non-illustrated tab switchover, using a scroll bar, or the like. Examinations to be confirmed may be selected from a non-illustrated examination list for switchable display.

The success/failure designation button 10202 does not necessarily have to have both a success (OK) button and a failure (not OK) button. For example, a “Not OK” checkbox 10306 or a non-illustrated OK/not OK toggle switch or the like may be used. In this case, it is possible to regard the designation as “success” (OK) if “failure” (not OK) is not ticked. A “Designate all as not OK” button 10307 may be used for changing the values of the success/failure designation button 10202 associated with all of the examinations respectively into “failure” (not OK) in a batch. The designation of the results of all of the examinations may be changed to “OK” when the “Designate all as not OK” button 10307 is touched again so as to clear the “failure” (not OK) designation of all of the examinations. Alternatively, a non-illustrated “Designate all as OK” button may be provided for this purpose. In a case where there exists an examination that has never been displayed in the confirmation screen 10200 when the “Designate all as not OK” button 10307 is touched, for the purpose of preventing the result of this unintended examination from being designated as “failure” (not OK), it will be convenient if a batch change into “failure” (not OK) is configured to be effected after displaying a message saying that this yet-to-be-displayed examination exists.

The method for designating “success/failure” for each examination is not limited to giving an instruction from the person conducting the examination. As described earlier in the first embodiment, the success/failure of an examination may be automatically determined at the time of acquiring the results of the examination. In a case where “failure” (not OK) is designated for an examination automatically, a button color or an icon color may be changed so that the person conducting the examination will be able to distinguish automatically designated “failure” (not OK) from manually designated “failure” (not OK). In a case where, for example, “failure” (not OK) is designated automatically for a yet-to-be-confirmed examination 10308 in FIG. 21B that is not displayed in the confirmation screen 10200(a) illustrated in FIG. 21A, for the purpose of preventing the operator's failure to notice this “failure” (not OK) designation, it will be effective to display a symbol and/or characters similar to the symbol and/or characters 10305 for letting the operator know that the yet-to-be-confirmed examination 10308 exists.

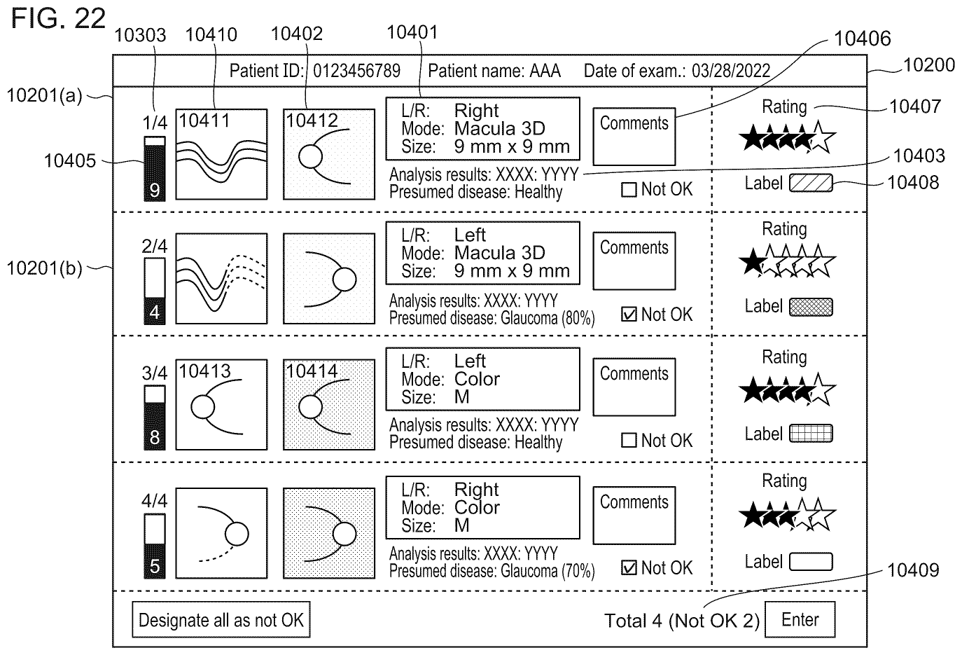

FIG. 22 illustrates an example in which accompanying supplementary information associated with each examination is displayed together therewith in the confirmation screen 10200. Each examination 10201 has any pieces of accompanying supplementary information the number of which is greater than or equal to zero besides a captured image 10410. The accompanying supplementary information is not specifically limited. For example, the accompanying supplementary information may include imaging parameters 10401 such as examination mode, size, and left/right eye information, examination quality score 10405, various analysis results 10403, and the like. For example, information such as patient ID, patient name, and date and time of examination may also be treated as accompanying supplementary information of the examination 10201. The captured image 10410 included in an examination may be a plurality of images and/or a plurality of types of images. For example, a tomographic volume image may be treated as one captured image. A plurality of types of captured images such as a tomographic image and an SLO image may be included in an examination. An image 10402 generated from the captured image 10410 included in an examination may also be treated as accompanying supplementary information of the examination 10201. The captured image 10410 itself included in an examination may also be treated as accompanying supplementary information of the examination 10201. The confirmation screen 10200 may display any pieces of accompanying supplementary information in an arranged manner or may display them in a switched manner.

Examples of the generated image 10402 include, specifically, a digital filter image 10414 generated from a fundus color photo 10413, a frontal image 10412 generated from an OCT volume image 10411, and the like. The generated frontal image 10412 may be an En-Face image obtained through any interlayer projection of the OCT volume image 10411. The generated image 10402 may be an image obtained by changing the luminance, contrast, tinge, shape, or the like of the captured image 10410, or may be an image obtained by superposing any character string, any figure, another image, or the like on an image. Display that shows a correspondence between the captured image 10410 and the generated image 10402 may be added.

Examples of the analysis values 10403 include, specifically, layer thickness, vascular density, optic disc size, the results of comparison with standard data, and the like. A disease may be estimated using pattern matching or machine learning, and estimated disease information such as disease location and disease name may be displayed as the analysis values 10403. The examination quality score 10405, etc. obtained through an evaluation conducted using a luminance, a contrast, and the like may also be treated as the analysis values 10403.

Besides the accompanying supplementary information affixed to the examination automatically, accompanying supplementary information that the operator can designate freely such as a rating 10407, a label 10408, and a comment box 10406 may be affixed thereto. For example, the quality score of the examination may be designated by using the rating 10407 from the viewpoint of the person conducting the examination, the examination 10201 may be put into a category by using the label 10408, or any comments may be added in the comment box 10406. The label 10408, for example, a color, a numeral, a symbol, any character string, or the like, is not specifically limited, and may be able to be designated in any number that is greater than or equal to zero.

The label and the comments may be used for, for example, registering a disease name, or may be used for meaning “Confirmation needed”. It may mean that the results are poor though image capturing was performed again.

The areas for displaying the examinations 10201 respectively may be configured to be able to be rearranged. For example, the row of the examination 10201(a) and the row of the examination 10201(b) illustrated in FIG. 22 may be permutable with each other by performing a drag operation or the like.

The examinations 10201 may be configured to be able to be rearranged by executing sorting using any value of the imaging parameters 10401, the rating 10407, or the like. The number of examinations 10409 may be displayed so that the operator can know the total number of images and the number of not-ok images at a glance. When the number of examinations 10409 is displayed, the display may be performed such that the number of not-OK images is shown in a case where there is one or more examinations for which “not OK” has been designated and the number of not-OK images is not shown in a case where there is no examination for which “not OK” has been designated.

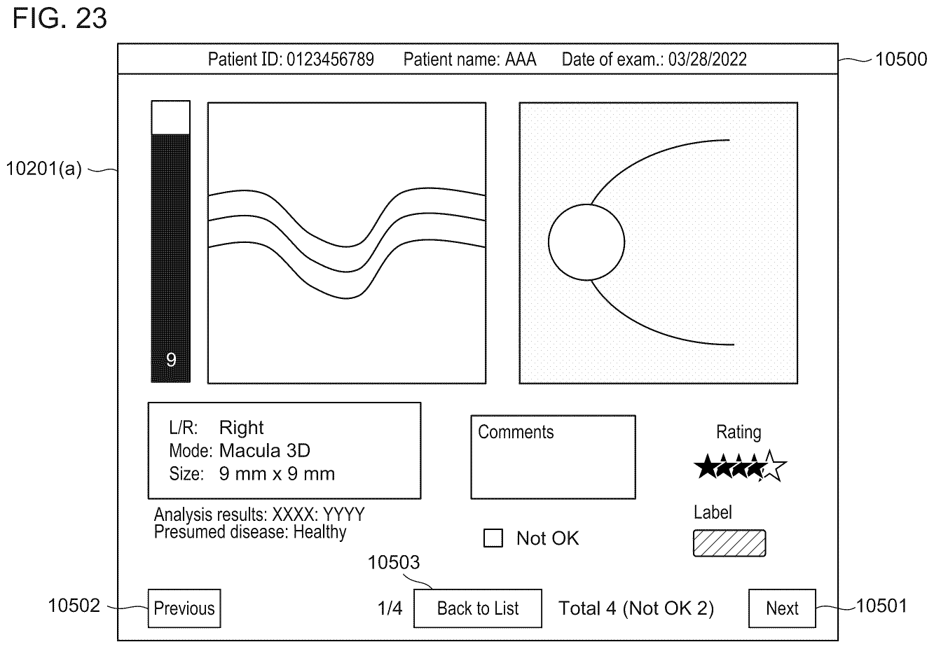

When the examination 10201(a) is selected in FIG. 22 or a non-illustrated enlarged display button is selected, the examination 10201(a) may be displayed on a confirmation screen (enlarged display screen) 10500 as illustrated in FIG. 23. At this time, a part or a whole of information that was displayed on the confirmation screen 10200 may be displayed on the confirmation screen (enlarged display screen) 10500, and/or information that was not displayed on the confirmation screen 10200 may be additionally displayed thereon. A Next button 10501 and a Back button 10502 may be used for switching the display to another examination. A Back to List button 10503 may be displayed so as to make it possible to return to the confirmation screen 10200. The confirmation screen (enlarged display screen) 10500 may be displayed by switching from the confirmation screen 10200. Alternatively, a pop-up dialogue for displaying the confirmation screen (enlarged display screen) 10500 may appear on the confirmation screen 10200.

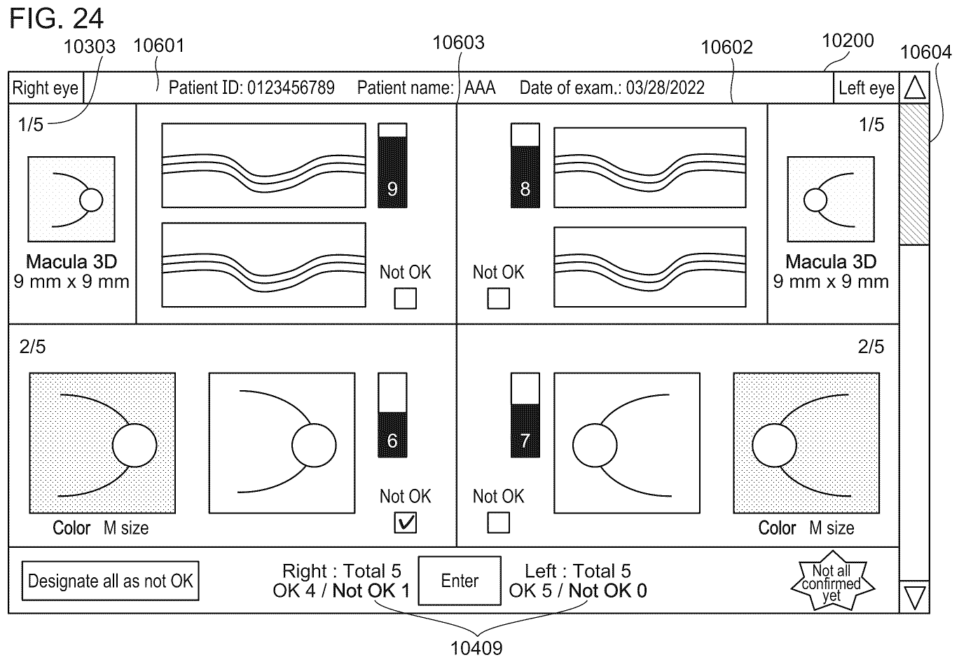

Next, another layout of the confirmation screen 10200 will now be described with reference to FIG. 24. In FIG. 24, display of the right-eye examination is presented at a display area 10601 located to the left of the center 10603 of the confirmation screen 10200, and display of the left-eye examination is presented at a display area 10602 located to the right of the center 10603 thereof. On the same row, examination images having been acquired in the same examination mode (those of a Macula 3D examination on the first row and fundus color photos on the second row) are displayed for the left eye and the right eye respectively. The layout of display such as arrangement and/or displayed information may be changed depending on the examination mode. In the present embodiment, values corresponding to the left eye and the right eye respectively are displayed for the examination numbering 10303 and for the number of examinations 10409; however, values counted without making a distinction between the left eye and the right eye may be displayed for them. In the present embodiment, the switching of the examination to be displayed is realized by a scroll bar 10604 in place of the Next button 10301 and the Previous button 10302 illustrated in FIGS. 21A and 21B. Any pieces of information and/or buttons may be arranged freely on the confirmation screen 10200 without being limited to the display example according to the present embodiment.

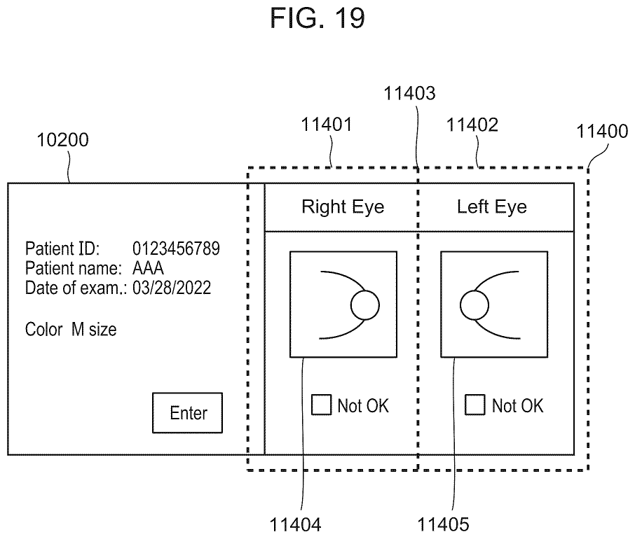

Though display of the left-eye examination and display of the right-eye examination are presented on the right side and on the left side respectively with respect to the center 10603 of the confirmation screen 10200 in FIG. 24, in a case where a main display area 11400 is biased in position toward the left or toward the right in the confirmation screen 10200 as illustrated in FIG. 19, display of the right-eye examination or display of an image 11404 included in the right-eye examination should be presented at a display area 11401 located to the left of the center 11403 of the main display area 11400, and display of the left-eye examination or display of an image 11405 included in the left-eye examination should be presented at a display area 11402 located to the right of the center 11403 thereof.

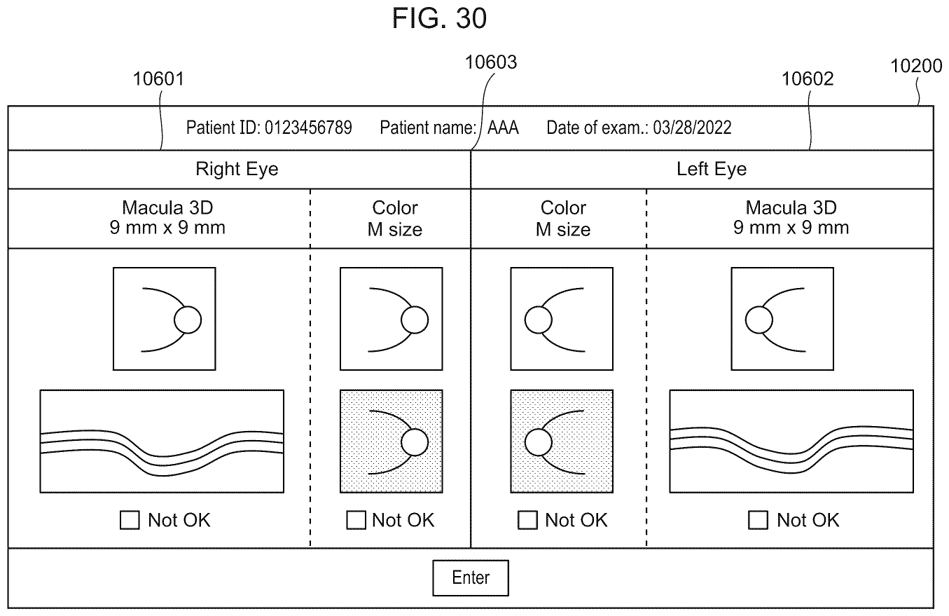

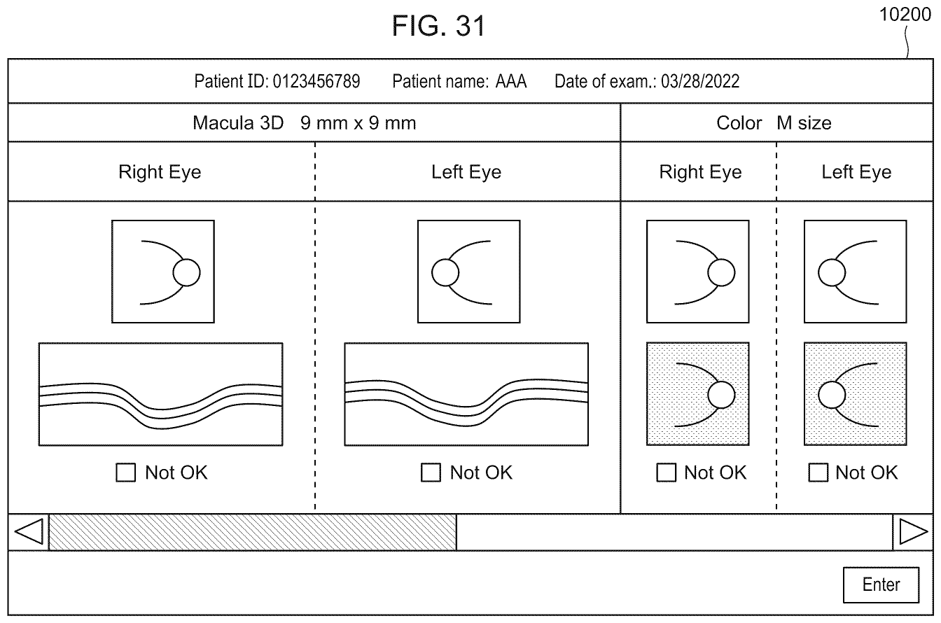

Though the display is presented in a 2×2 layout (the left-eye examination and the right-eye examination×two rows) in the present embodiment, depending on the number of examinations to be displayed, the display may be presented in any other layout such as a 2×1 layout (the left-eye examination and the right-eye examination×one row) or in a 2×3 layout (the left-eye examination and the right-eye examination×three rows). The number of examinations displayable on one row is not limited to two examinations for the left eye and the right eye; a 4×1 layout (two examinations for the left eye and two examinations for the right eye are arranged horizontally in one row) or the like such as an example illustrated in FIG. 30 or an example illustrated in FIG. 31 may be adopted instead. In the example illustrated in FIG. 30, display of a plurality of right-eye examinations is presented at the display area 10601 located to the left of the center 10603 of the confirmation screen 10200, and display of a plurality of left-eye examinations is presented at the display area 10602 located to the right of the center 10603 thereof. In the example illustrated in FIG. 31, the confirmation screen 10200 is displayed in a categorized manner according to the same examination mode, and display of the right-eye examination is presented to the left of display of the left-eye examination in the same examination mode.

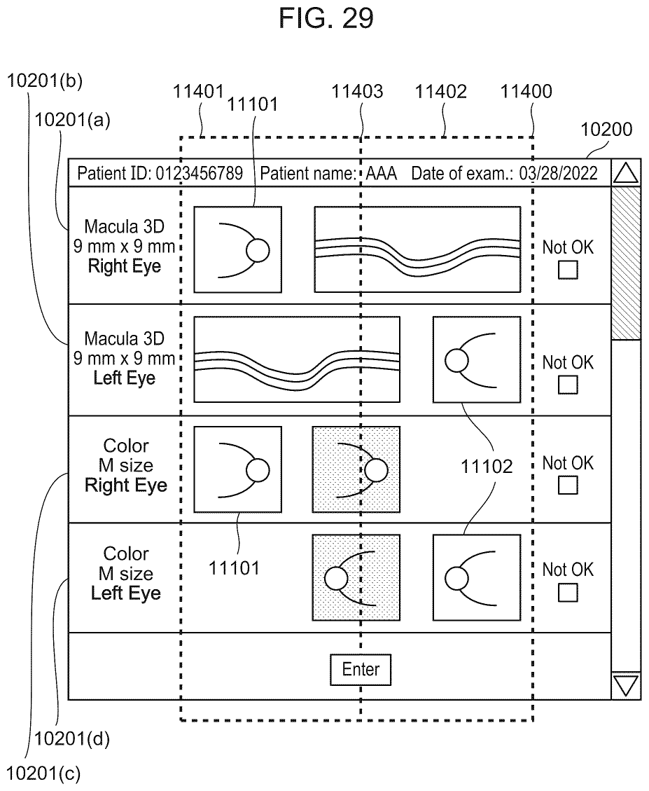

FIG. 29 illustrates an example in which left/right-eye examinations are arranged vertically. Main frontal images (for the right eye) 11101 included in right-eye examinations 10201(a) and 10201(c) are displayed at the display area 11401 located to the left of the center 11403 of the main display area 11400, and main frontal images (for the left eye) 11102 included in left-eye examinations 10201(b) and 10201(d) are displayed at the display area 11402 located to the right of the center 11403 thereof. Even in a case where left/right-eye examinations cannot be arranged horizontally, this makes it possible for the operator to identify the left/right-eye examinations intuitively and confirm them easily. It is sufficient as long as the left/right-eye examinations are identifiable, and the means for identification is not limited to the above-described layout; instead, different icons may be assigned to the left/right-eye examinations, or the display size of them may differ. The left/right-eye examinations may be made identifiable by adopting different background colors, different character font types or sizes, or the like for the left/right-eye examinations.

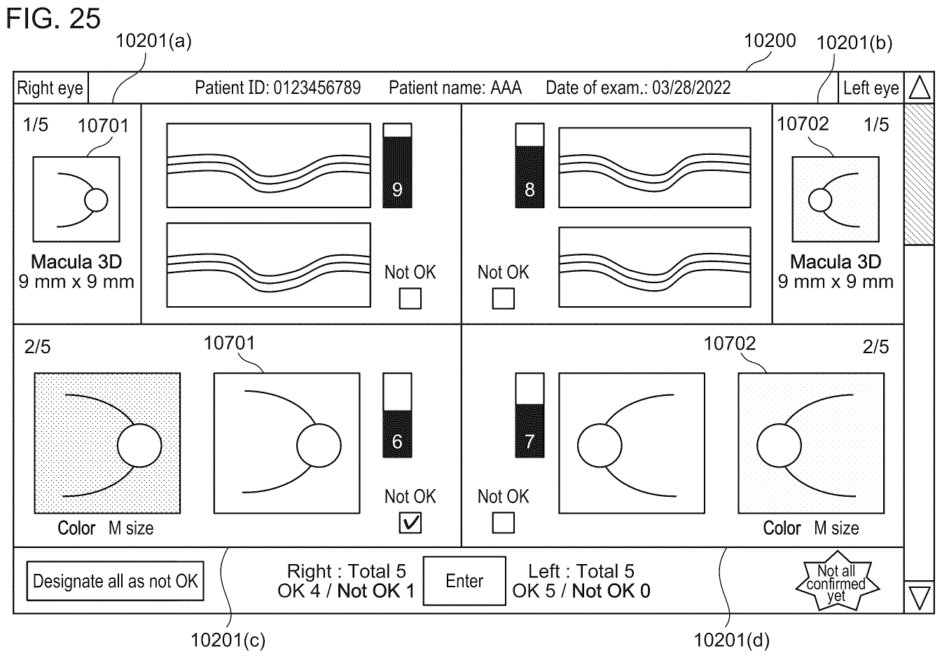

Accompanying supplementary information of another examination may be displayed in an associated manner in the display area of each examination as illustrated in FIG. 25. For example, a fundus color photo 10701 of the examination 10201(c) may be displayed instead of displaying a frontal image of the examination 10201(a). A frontal image 10702 of the examination 10201(b) may be displayed instead of displaying a digital filter image of the examination 10201(d). Examples of such a frontal image for replacement include an En-Face image, an SLO image, a fundus color photo, a digital filter image, and the like. Though each image 10701, 10702 is displayed as a replacement in the present embodiment, the image may be displayed in a superimposed manner or may be displayed in another area adjacently. The accompanying supplementary information of another examination displayed in the display area of each examination is not limited to an image but may be information such as an analysis value. The accompanying supplementary information of the opposite eye may be displayed in an associated manner. An analysis of the difference between the left eye and the right eye may be displayed. An instruction for a luminance change, a contrast change, a color adjustment, or the like for any examination may be applied to another examination to be displayed.

### Fifth Embodiment

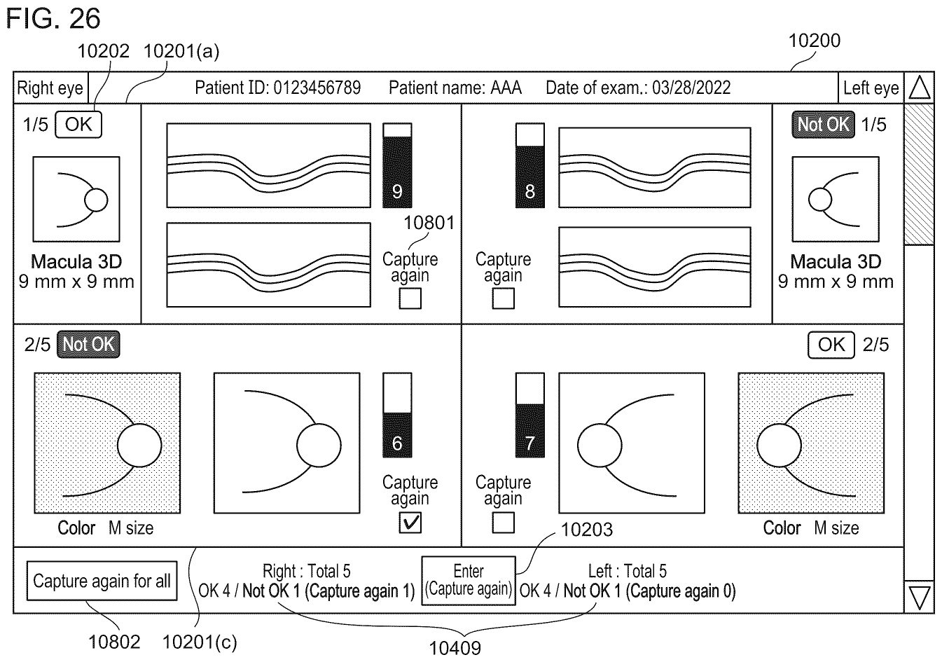

A variation example of the fourth embodiment will now be described. In FIG. 26, the confirmation screen 10200 includes, besides the success/failure designation button 10202, a capturing-again instruction button 10801 associated with each examination; on this screen, for each examination individually, the operator is able to give an instruction as to whether to perform image capturing again. When the Enter button 10203 is selected, image capturing is performed again for the examination(s) for which it is instructed to perform image capturing again. It is recommendable to, at this time, make the processing performed in response to pressing the Enter button 10203 different depending on whether or not there is any examination for which it is instructed to perform image capturing again. For example, in a case where there is one or more examinations for which it is instructed to perform image capturing again, image capturing may be performed again instead of finalizing the success/failure designation in response to pressing the Enter button 10203, and the finalizing of the success/failure designation may be performed only if there is no examination for which it is instructed to perform image capturing again. Depending on whether or not there is any examination for which it is instructed to perform image capturing again and depending on the number thereof, the display content of the Enter button 10203 may be changed. For example, the Enter button 10203 may be displayed as “Enter” in a case where there is no examination for which it is instructed to perform image capturing again. The Enter button 10203 may be displayed as “Capturing again”, or the number of examinations for which image capturing is performed again may be displayed, in a case where there is one or more examinations for which it is instructed to perform image capturing again.

The success/failure designation button 10202 may be linked to the value of the capturing-again instruction button 10801. For example, when an instruction to perform image capturing again is given via the capturing-again instruction button 10801 associated with a certain examination 10201(a), the success/failure designation button 10202 associated with this examination 10201(a) may be changed to “failure” (not OK). Conversely, when the success/failure designation button 10202 associated with a certain examination 10201(a) is changed to “failure” (not OK), the capturing-again instruction checkbox 10801 associated with this examination 10201(a) may be changed to “Capture again”. Of course, linking the capturing-again instruction button 10801 and the success/failure designation button 10202 with each other is not necessarily needed. The values of them may be changeable independently of each other. The link may be in one direction only. The success/failure designation button 10202 may be omitted, and the success/failure designation may be performed using the capturing-again instruction button 10801. In this case, each icon or each label that indicates the current status of success/failure designation may be displayed in place of the success/failure designation button 10202, and the success/failure designation status may be switched using the capturing-again instruction button 10801. The values of all of the capturing-again instruction buttons 10801 may be changed in a batch by pressing a “Capturing again for all” button 10802. The number of examinations for which it is instructed to perform image capturing again may also be displayed together at the number of examinations 10409.

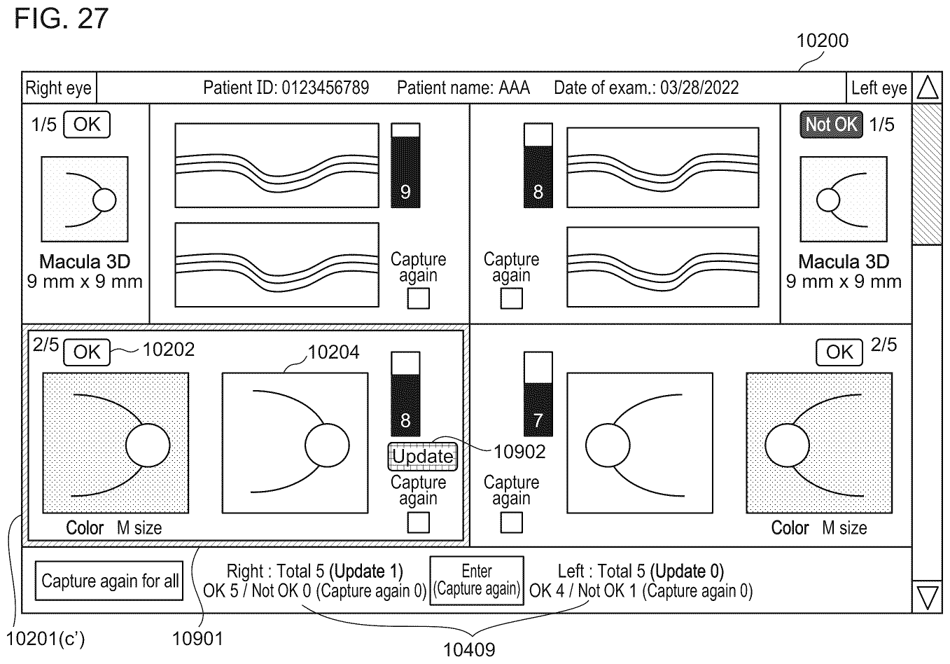

Next, FIG. 27 illustrates an example of the confirmation screen 10200 after performing image capturing again for the examination 10201(c) for which it is instructed to perform image capturing again in FIG. 26. In FIG. 27, updated display with replacement of the examination 10201(c) by a captured-again examination 10201(c′) is presented. To enable the operator to know that this display is updated display, for example, the line thickness or color of a frame 10901 may be changed and/or an Update icon 10902 may be displayed. It will be convenient if the number of updated examinations is also displayed together at the number of examinations 10409. The examination before the updating is not displayed in the present embodiment. However, the examination before the updating may be displayed adjacently or may be displayed in a switched manner. In a case where the success/failure of the examination before the updating is configured to be changeable, control may be performed such that not both of the examination before the updating and the examination after the updating are designated as “success (OK)”. For example, if the examination 10201(c) before the updating is better than the captured-again examination 10201(c′), the designation of the captured-again examination 10201(c′) should be changed to “failure” (not OK) when the designation of the examination 10201(c) before the updating is changed from “failure” (not OK) to “success (OK)”. In a case where both the examination before the updating and the captured-again examination are designated as “failure” (not OK), that is, in a case where there is no examination designated as “success (OK)”, the capturing-again instruction checkbox 10801 may be changed to “Capture again”.

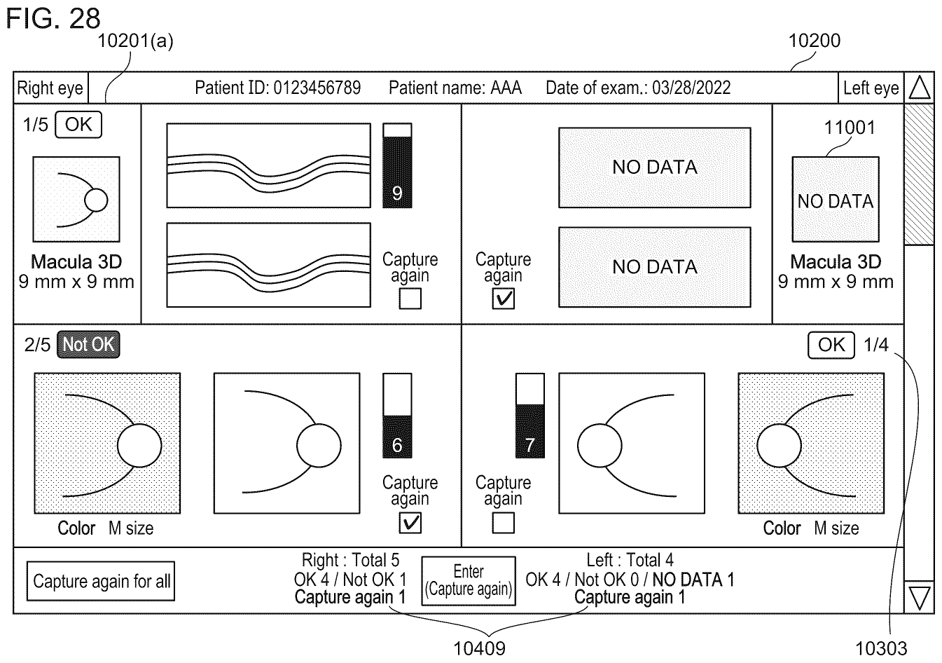

Next, with reference to FIG. 28, a display example for a case where the corresponding examination does not exist will now be described. When a plurality of acquired examinations is displayed with correspondences to the left eye and the right eye on an examination-mode-by-examination-mode basis, the opposite-eye examination corresponding to a certain examination does not always exist. For example, in a case where a left-eye examination 11001 corresponding to the right-eye examination 10201(a) having been acquired in a Macula 3D mode does not exist, it will be convenient if display is presented to the effect that the examination 11001 does not exist. It may be configured such that an image can be captured anew by giving a capturing-again instruction in a case where the examination 11001 does not exist. The examination 11001 that does not exist may be included in, or not included in, the examination numbering 10303 and the number of examinations 10409. In this example, the examination 11001 that does not exist is not included in the examination numbering 10303 but is displayed at the number of examinations 10409.

### Sixth Embodiment

Though examples of a confirmation screen have been described in the fourth and fifth embodiments, a report screen may be displayed in substantially the same layout as that of the confirmation screen 10200. For example, a combination of a plurality of examinations displayed on the confirmation screen 10200 may be stored, and the same combination of the examinations may be displayed together on a report screen, too. The report screen may be displayed in different states of display for the left-eye examination and the right-eye examination so as to make the left/right-eye examination identifiable, similarly to the confirmation screen 10200 having been described in the fourth embodiment. When the report screen is displayed in this manner, each display item and each operation button, etc. of the confirmation screen 10200 may be in a non-display state. Conversely, the report screen may be displayed with any display item and/or any operation button added thereto.

### First Variation Example

In an examination protocol according to various above-described embodiments, various kinds of guidance may be automatically provided by voice using the voice output unit 350. For example, during-alignment guidance, left/right-eye movement guidance, capturing start timing guidance, and/or subject's eye wink-prompting guidance may be provided by voice. Providing guidance by voice makes it possible to proceed with image capturing without the operator having to give an explanation to the subject.

### Second Variation Example

In an examination protocol according to various above-described embodiments, it has been assumed that image capturing is performed for the right eye first, and the left eye next, in describing an examination flow of capturing images of both eyes through examinations included in the examination protocol successively. However, this does not imply any limitation. Image capturing may be performed for the left eye first, and the right eye next. The operator may be allowed to set which one of the left eye and the right eye to start image capturing from. This makes it possible for each facility to set its own way of image capturing.

### Third Variation Example

In various above-described embodiments, a finger-based touch operation has been described. However, this does not imply any limitation. An input device such as a touch pen or a mouse may be used for performing an operation. In this case, the operator can perform both a touch operation using a finger and a click operation using a mouse, and an input is acceptable from both.

### Fourth Variation Example

In an examination protocol according to various above-described embodiments, when images are captured through examinations included in the examination protocol successively, a function of automatic pausing may be provided, without being limited to manual pausing by the operator. For example, there may be a pause when the apparatus is controlled from the right eye to the left eye. Restarting may be performed in response to an instruction given by the operator. Restarting may be performed automatically using a timer or the like. There may be a pause before/after fundus imaging. For example, in a case where pausing is performed before fundus imaging, the process may be paused automatically, and the timing of capturing an image may be left up to the operator. In a case where pausing is performed after fundus imaging, the process may be restarted after waiting for the returning of the subject's eye from pupil contraction.

Techniques according to the present disclosure may include the following configurations and methods.

### Configuration 1

An ophthalmic apparatus may include:
an examination unit configured to examine an eye of an examinee;
a storage unit configured to store a plurality of examination protocols, each of the plurality of examination protocols including a plurality of examinations; and
a control unit configured to control a display unit such that information that represents a plurality of examinations included in each of at least two examination protocols among the plurality of examination protocols stored is displayed on a protocol-by-protocol basis for the at least two examination protocols, and configured to control the examination unit in accordance with one examination protocol selected according to an instruction given from an examiner from among the at least two examination protocols.

### Configuration 2

In the ophthalmic apparatus according to Configuration 1, the information that represents the plurality of examinations may be information that represents the plurality of examinations such that a sequential order of the plurality of examinations is recognizable.

### Configuration 3

In the ophthalmic apparatus according to Configuration 1 or 2, a scanning pattern corresponding to an examination regarding OCT imaging included in the examination protocol may be any of 3D scan, radial scan, cross scan, multi-cross scan, circle scan, and line scan, and information that represents the scanning pattern may be included in the information that represents the plurality of examinations.

### Configuration 4

An ophthalmic apparatus may include:
an examination unit configured to examine an eye of an examinee;
a storage unit configured to store a plurality of examination protocols, each of the plurality of examination protocols including at least one examination; and
a control unit configured to control a display unit such that information that represents at least one examination included in each of at least two examination protocols among the plurality of examination protocols stored is displayed on a protocol-by-protocol basis for the at least two examination protocols, and configured to control the examination unit in accordance with one examination protocol selected according to an instruction given from an examiner from among the at least two examination protocols.

### Configuration 5

In the ophthalmic apparatus according to any one of Configurations 1 to 4, the control unit may control the display unit such that information that shows status regarding the plurality of examinations is displayed when the plurality of examinations included in the selected one examination protocol is executed successively.

### Configuration 6

An ophthalmic apparatus may include:
an examination unit configured to examine an eye of an examinee;
a storage unit configured to store a plurality of examination protocols, each of the plurality of examination protocols including a plurality of examinations; and
a control unit configured to control the examination unit in accordance with one examination protocol selected according to an instruction given from an examiner from among the plurality of examination protocols stored, and configured to control a display unit such that information that shows status regarding the plurality of examinations is displayed when the plurality of examinations included in the selected one examination protocol is executed successively.

### Configuration 7

In the ophthalmic apparatus according to Configuration 5 or 6, the information that shows the status may be information that shows a current examination progress status and represents any one of capturing done, under capturing, and waiting for capturing in the plurality of examinations included in the selected one examination protocol.

### Configuration 8

In the ophthalmic apparatus according to any one of Configurations 1 to 7, the examination unit may be capable of performing at least one image capturing among fundus imaging, anterior eye segment imaging, fluorescent fundus imaging, fundus OCT imaging, and anterior eye segment OCT imaging.

### Configuration 9

An information processing apparatus may include:
a control unit configured to control a display unit such that an examination result for a left eye and an examination result for a right eye that are included in a plurality of examination results obtained by examining the eyes of an examinee in accordance with an examination protocol that includes an examination of the left eye and an examination of the right eye, and configured to control the display unit such that display information is displayed for receiving an entering as to success-or-failure designation of the plurality of examinations according to an instruction given from an examiner.

### Configuration 10

In the information processing apparatus according to Configuration 9, the control unit may control the display unit such that the examination result for the left eye and the examination result for the right eye are displayed in different states of display in terms of at least one of different color, different font, different size, and different layout.

### Configuration 11

In the information processing apparatus according to Configuration 9 or 10, the control unit may control the display unit such that display of the examination of the right eye is presented to the left of the center of a display area and such that display of the examination of the left eye is presented to the right of the center of the display area.

### Configuration 12

In the information processing apparatus according to any one of Configurations 9 to 11, the control unit may control the display unit such that a frontal fundus image included in the examination of the right eye is displayed to the left of the center of a display area and such that a frontal fundus image included in the examination of the left eye is displayed to the right of the center of the display area.

### Configuration 13

In the information processing apparatus according to any one of Configurations 9 to 12, the control unit may control the display unit such that examinations having been acquired in the same examination mode for the left eye and the right eye are displayed in a corresponding manner.

### Configuration 14

In the information processing apparatus according to any one of Configurations 9 to 13, the control unit may control the display unit such that the examination of the right eye is displayed to the left of the examination of the left eye having been acquired in the same examination mode.

### Configuration 15

In the information processing apparatus according to any one of Configurations 9 to 14, the control unit may control the display unit such that a frontal fundus image included in the examination of the right eye is displayed to the left of a frontal fundus image included in the examination of the left eye of the same examination mode.

### Configuration 16

In the information processing apparatus according to any one of Configurations 9 to 15, the control unit may control the display unit such that, in a case where an examination of one eye having been acquired in the same examination mode for any examination does not exist, display is presented to the effect that the examination of the one eye of the same examination mode does not exist.

### Configuration 17

In the information processing apparatus according to any one of Configurations 9 to 16, the control unit may control the display unit such that information regarding the number of examinations corresponding to each of the examination of the left eye and the examination of the right eye that are included in the plurality of examinations is displayed.

### Configuration 18

In the information processing apparatus according to any one of Configurations 9 to 17, the control unit may control the display unit such that information regarding the sequential order of examinations corresponding respectively to examination results that are displayed among the results of the plurality of examinations is displayed. The control unit may control the display unit such that the examination results that are displayed among the results of the plurality of examinations are changed to results of other examinations according to an instruction given from the examiner (for example, an instruction to a scroll bar located at the right side of a confirmation screen).

### Configuration 19

In the information processing apparatus according to any one of Configurations 9 to 18, the control unit may control the display unit such that at least one of an image included in another examination and accompanying supplementary information is displayed in combination with any examination. The image included in another examination may be and of an En-Face image, an SLO image, and a fundus color photo.

### Configuration 20

In the information processing apparatus according to any one of Configurations 9 to 19, the control unit may control the display unit such that, in a case where any examination that has not been confirmed yet exists among the plurality of examinations, display is presented to the effect that the examination that has not been confirmed yet exists.

### Configuration 21

The information processing apparatus according to Configuration 20 may be configured not to be able to perform processing regarding the entering, even upon receiving the entering as to the success-or-failure designation of the plurality of examinations according to the instruction given from the examiner, in the case where the examination that has not been confirmed yet exists.

### Configuration 22

In the information processing apparatus according to any one of Configurations 9 to 21, the control unit may control the display unit such that the results of the plurality of examinations are displayed in a layout that differs depending on a number of examinations corresponding to the plurality of examinations.

### Configuration 23

In the information processing apparatus according to any one of Configurations 9 to 22, the control unit may control the display unit such that the results of the plurality of examinations are displayed in a layout that differs depending on an examination mode (examination type) corresponding to the plurality of examinations.

### Configuration 24

In the information processing apparatus according to any one of Configurations 9 to 23, the control unit may control the display unit such that display information is displayed for receiving a designation as to whether the result of each of the plurality of examinations is a success or a failure according to an instruction given from the examiner.

### Configuration 25

In the information processing apparatus according to any one of Configurations 9 to 24, the control unit may control the display unit such that display information that indicates whether the result of each of the plurality of examinations is a success or a failure and is obtained by performing automatic determination using the result of each of the plurality of examinations is displayed.

### System

A system may include:
an ophthalmic apparatus that includes an examination unit configured to examine an eye of an examinee (for example, the ophthalmic apparatus according to any one of Configurations 1 to 8); and
an information processing apparatus according to any one of Configurations 9 to 25 connected to the ophthalmic apparatus communicably.

### Other Configurations

In response to the enter instruction, information that indicates whether the result of each of the plurality of examinations is a success or a failure may be affixed as accompanying supplementary information. Different processing may be applied to the results of examinations designated as successful and the results of examinations designated as not successful. In response to the enter instruction, control may be performed to store the results of examinations designated as successful and not to store the results of examinations designated as not successful. In response to the enter instruction, control may be performed to transfer the results of examinations designated as successful to an external system and not to transfer the results of examinations designated as not successful to the external system. The control unit may control the display unit such that display information is displayed for receiving an instruction for performing image capturing again for any examination among the plurality of examinations. The enter instruction of finalizing the designation of the failure may be given by giving the instruction for performing image capturing again. The instruction for performing image capturing again may be given by giving the enter instruction of finalizing the designation of the failure. The state of display may differ depending on whether or not there is an examination for which the instruction for performing image capturing again is given. The control unit may control the display unit such that, in place of the examination for which the instruction for performing image capturing again is given, the result of the captured-again examination is displayed. The control unit may control the display unit such that, among the plurality of examinations, the results of the captured-again examinations and the results of other examinations are displayed in an identifiable format from each other. The control unit may control the display unit such that the results of the plurality of examinations are displayed on a confirmation screen. The control unit may control the display unit such that the results of the plurality of examinations are displayed on a report screen in substantially the same layout as that of the confirmation screen.

### Method 1

The techniques according to the present disclosure may be embodied as: A method of controlling an ophthalmic apparatus, the ophthalmic apparatus including an examination unit configured to examine an eye of an examinee and a storage unit configured to store a plurality of examination protocols, each of the plurality of examination protocols including a plurality of examinations, the method comprising:
controlling a display unit such that information that represents a plurality of examinations included in each of at least two examination protocols among the plurality of examination protocols stored is displayed on a protocol-by-protocol basis for the at least two examination protocols; and
controlling the examination unit in accordance with one examination protocol selected according to an instruction given from an examiner from among the at least two examination protocols.

### Method 2

The techniques according to the present disclosure may be embodied as: A method of controlling an ophthalmic apparatus, the ophthalmic apparatus including an examination unit configured to examine an eye of an examinee and a storage unit configured to store a plurality of examination protocols, each of the plurality of examination protocols including a plurality of examinations, the method comprising:
controlling the examination unit in accordance with one examination protocol selected according to an instruction given from an examiner from among the plurality of examination protocols stored; and
controlling a display unit such that information that shows status regarding the plurality of examinations is displayed when the plurality of examinations included in the selected one examination protocol is executed successively.

### Method 3

The techniques according to the present disclosure may be embodied as: A method of controlling an ophthalmic apparatus, the ophthalmic apparatus including an examination unit configured to examine an eye of an examinee and a storage unit configured to store a plurality of examination protocols, each of the plurality of examination protocols including at least one examination, the method comprising:
controlling a display unit such that information that represents at least one examination included in each of at least two examination protocols among the plurality of examination protocols stored is displayed on a protocol-by-protocol basis for the at least two examination protocols; and
controlling the examination unit in accordance with one examination protocol selected according to an instruction given from an examiner from among the at least two examination protocols.

### Method 4

The techniques according to the present disclosure may be embodied as: A method of controlling an information processing apparatus, the method comprising:
controlling a display unit such that an examination result for a left eye and an examination result for a right eye are displayed in different states of display, the examination result for the left eye and the examination result for the right eye being included in a plurality of examination results obtained by examining the eyes of an examinee in accordance with an examination protocol that includes an examination of the left eye and an examination of the right eye; and
controlling the display unit such that display information is displayed for receiving an entering as to success-or-failure designation of the plurality of examinations according to an instruction given from an examiner.

### Program and Storage Medium

The technique according to the present disclosure may be embodied as: A program causing a computer to execute the control method according to any one of Methods 1 to 4, or a non-transitory computer-readable storage medium storing the program.

According to one of the techniques disclosed herein, it is possible to enhance the usability of an ophthalmic apparatus.

The present disclosure is not limited to the embodiments having been described above, and various alterations and modifications can be made without departing from the spirit and scope of the present disclosure. Claims are appended hereto so as to make the claimed scope of the present disclosure public.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An ophthalmic apparatus, comprising:
an examination unit configured to examine an eye of an examinee;
a storage unit configured to store a plurality of examination protocols, each of the plurality of examination protocols including a plurality of examinations; and
a control unit configured to control a display unit such that information that represents a plurality of examinations included in each of at least two examination protocols among the plurality of examination protocols stored is displayed on a protocol-by-protocol basis for the at least two examination protocols, and configured to control the examination unit in accordance with one examination protocol selected according to an instruction given from an examiner from among the at least two examination protocols.

2. The ophthalmic apparatus according to claim 1, wherein
the information that represents the plurality of examinations is information that represents the plurality of examinations such that a sequential order of the plurality of examinations is recognizable.

3. The ophthalmic apparatus according to claim 1, wherein
a scanning pattern corresponding to an examination regarding OCT imaging included in the examination protocol is any of 3D scan, radial scan, cross scan, multi-cross scan, circle scan, and line scan, and
information that represents the scanning pattern is included in the information that represents the plurality of examinations.

4. An ophthalmic apparatus, comprising:
an examination unit configured to examine an eye of an examinee;
a storage unit configured to store a plurality of examination protocols, each of the plurality of examination protocols including at least one examination; and
a control unit configured to control a display unit such that information that represents at least one examination included in each of at least two examination protocols among the plurality of examination protocols stored is displayed on a protocol-by-protocol basis for the at least two examination protocols, and configured to control the examination unit in accordance with one examination protocol selected according to an instruction given from an examiner from among the at least two examination protocols.

5. The ophthalmic apparatus according to claim 1, wherein
the control unit controls the display unit such that information that shows status regarding the plurality of examinations is displayed when the plurality of examinations included in the selected one examination protocol is executed successively.

6. An ophthalmic apparatus, comprising:
an examination unit configured to examine an eye of an examinee;
a storage unit configured to store a plurality of examination protocols, each of the plurality of examination protocols including a plurality of examinations; and
a control unit configured to control the examination unit in accordance with one examination protocol selected according to an instruction given from an examiner from among the plurality of examination protocols stored, and configured to control a display unit such that information that shows status regarding the plurality of examinations is displayed when the plurality of examinations included in the selected one examination protocol is executed successively.

7. The ophthalmic apparatus according to claim 5, wherein
the information that shows the status is information that shows a current examination progress status and represents any one of capturing done, under capturing, and waiting for capturing in the plurality of examinations included in the selected one examination protocol.

8. The ophthalmic apparatus according to claim 1, wherein
the examination unit is capable of performing at least one image capturing among fundus imaging, anterior eye segment imaging, fluorescent fundus imaging, fundus OCT imaging, and anterior eye segment OCT imaging.

9. A method of controlling an ophthalmic apparatus, the ophthalmic apparatus including an examination unit configured to examine an eye of an examinee and a storage unit configured to store a plurality of examination protocols, each of the plurality of examination protocols including a plurality of examinations, the method comprising:
controlling a display unit such that information that represents a plurality of examinations included in each of at least two examination protocols among the plurality of examination protocols stored is displayed on a protocol-by-protocol basis for the at least two examination protocols; and
controlling the examination unit in accordance with one examination protocol selected according to an instruction given from an examiner from among the at least two examination protocols.

10. A method of controlling an ophthalmic apparatus, the ophthalmic apparatus including an examination unit configured to examine an eye of an examinee and a storage unit configured to store a plurality of examination protocols, each of the plurality of examination protocols including a plurality of examinations, the method comprising:
controlling the examination unit in accordance with one examination protocol selected according to an instruction given from an examiner from among the plurality of examination protocols stored; and
controlling a display unit such that information that shows status regarding the plurality of examinations is displayed when the plurality of examinations included in the selected one examination protocol is executed successively.

11. A method of controlling an ophthalmic apparatus, the ophthalmic apparatus including an examination unit configured to examine an eye of an examinee and a storage unit configured to store a plurality of examination protocols, each of the plurality of examination protocols including at least one examination, the method comprising:
controlling a display unit such that information that represents at least one examination included in each of at least two examination protocols among the plurality of examination protocols stored is displayed on a protocol-by-protocol basis for the at least two examination protocols; and
controlling the examination unit in accordance with one examination protocol selected according to an instruction given from an examiner from among the at least two examination protocols.

12. A non-transitory computer-readable storage medium storing a program causing a computer to execute the method according to claim 9.

13. The ophthalmic apparatus according to claim 1, wherein
the plurality of examination protocols includes a left-eye-and-right-eye examination protocol that includes an examination of a left eye and an examination of a right eye,
the control unit controls the display unit such that an examination result for the left eye and an examination result for the right eye that are included in a plurality of examination results obtained by examining the eyes of the examinee in accordance with the left-eye-and-right-eye examination protocol are displayed in different states of display, and
the control unit controls the display unit such that display information is displayed for receiving an entering as to success-or-failure designation of the plurality of examinations according to an instruction given from the examiner.

14. The ophthalmic apparatus according to claim 13, wherein
the control unit controls the display unit such that the examination result for the left eye and the examination result for the right eye are displayed in different states of display in terms of at least one of different color, different font, different size, and different layout.

15. The ophthalmic apparatus according to claim 13, wherein
the control unit controls the display unit such that the results of the plurality of examinations are displayed in a layout that differs depending on a number of examinations corresponding to the plurality of examinations.

16. The ophthalmic apparatus according to claim 13, wherein
the control unit controls the display unit such that the results of the plurality of examinations are displayed in a layout that differs depending on an examination mode corresponding to the plurality of examinations.

17. A non-transitory computer-readable storage medium storing a program causing a computer to execute the method according to claim 10.

18. A non-transitory computer-readable storage medium storing a program causing a computer to execute the method according to claim 11.
